(19) United States
(12) Patent Application Publication
(10) Pub. No.: US 2015/0093773 A1
(43) Pub. Date: Apr. 2, 2015

(54) METHOD OF INSERTING DNA FRAGMENTS INTO GENOME BY USING MARKER GENE EXPRESSION

(71) Applicants: Samsung Electronics Co., Ltd., Suwon-si (KR); Industry-Academic Cooperation Foundation, Yonsei University, Seoul (KR)

(72) Inventors: Yukyung Jung, Hwaseong-si (KR); Duhee Bang, Seoul (KR); Jinhwan Park, Suwon-si (KR); Hanna Seo, Seoul (KR); Jaehwan Jung, Seoul (KR); Kwangmyung Cho, Seongnam-si (KR)

(21) Appl. No.: 14/503,786

(22) Filed: Oct. 1, 2014

(30) Foreign Application Priority Data
Oct. 1, 2013 (KR) 10-2013-0117493
Sep. 29, 2014 (KR) 10-2014-0130327

(51) Int. Cl. C12N 15/70

(52) U.S. Cl. CPC C12N 15/70
USPC 435/29; 435/455; 435/468; 435/471; 435/446

(57) ABSTRACT

Provided is a method of inserting a target sequence into a genome of a cell through positive selection or negative selection based on whether a marker is expressed.

### RELATED APPLICATION

This application claims the benefit of Korean Patent Application Nos. 10-2013-0117493, filed on Oct. 1, 2013, and 10-2014-0130327, filed on Sep. 29, 2014, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: 29,303 ASCII (Text) file named “718600_ST25.TXT” created Sep. 30, 2014.

### BACKGROUND

1. Field

The present disclosure relates to a method of inserting a DNA fragment by using a marker gene.

2. Description of the Related Art

The technology of mutating a genome to produce a genome-mutated organism having a desired trait is a classical molecular biological study based on a modified genome. Recently, however, the technology has been often used to produce biofuel or biochemical materials by using bacteria. Such a genome mutation may insert, substitute, or delete not only a few nucleotides but also a DNA fragment having the size of a gene.

Generally, a DNA fragment may be inserted to a genome of a Gram negative bacterium by using Red proteins (exo, beta, gamma) of a lambda bacteriophage. This requires a linearly designed DNA fragment to be inserted, and the DNA fragment is inserted to a cell by such methods as electroporation. To be inserted to a specific site on the genome, a DNA fragment includes a flanking sequence, which is a polynucleotide sequence of about 50 base pair (bp) on both sides of the specific insertion site and included in the DNA fragment in the directions of the two ends of the DNA fragment. The transferred DNA fragment is converted from a double strand to a single strand by the activity of an exo protein, which is one of the Red proteins, and then protected by being bound to a beta protein in order not to be degraded in the cell.

The single-stranded DNA fragment is inserted to the genome in the form of an Okazaki fragment during the genome replication process of the cell. Therefore, as the length of the DNA fragment is increased, the conversion of the double-stranded DNA fragment into a single-stranded DNA fragment by the exo protein becomes less efficient, and this leads directly to the decrease of the efficiency of inserting the entire DNA fragment.

Another method employs a DNA fragment including a marker gene, inserting the entire resulting DNA fragment to a genome, and searching the DNA fragment by using the inserted marker gene has been suggested. However, the method is not efficient, because the size of the DNA fragment to be inserted is rather increased. In addition, in selecting a marker gene, an antibiotic resistance gene allowing only positive selection is generally used. Therefore, a new antibiotic resistance gene allowing a positive selection should be used each time when a DNA fragment is inserted. However, it is practically limited to use a new antibiotic resistance gene each time, since the kinds of antibiotic resistance gene are limited.

Therefore, there is a need for a method of using a marker gene preexisting in a genome, to reduce the size of the DNA fragment which is be inserted to maximize the insertion efficiency and conveniently search for a modified gene.

### SUMMARY

Provided is a method of inserting a target sequence into a genome of a cell. In one aspect, the method comprises inserting a target sequence into a genome of a cell, by introducing to the cell a first polynucleotide comprising, in a direction from 5′ to 3′, a first flanking sequence, a first region including a first target sequence, and a second flanking sequence. The genome of the cell comprises, in a direction from 5′ to 3′, a second region comprising a transcription regulation site of a marker encoding region, and a third region comprising a marker encoding region. The first flanking sequence is homologous with at least two consecutive nucleotides in the second region of the genome of the cell, at least two consecutive nucleotides in a 5′ direction from a 5′-end of the second region, or a combination thereof, and the second flanking sequence is homologous with at least two consecutive nucleotides in the second region of the genome of the cell, at least two consecutive nucleotides in a 3′ direction from a 5′-end of the third region, or a combination thereof. By inserting the first polynucleotide into the cell, the first region of the first polynucleotide is introduced into the genome to provide a first region of the genome. Recombinant cells can be selected based on a lack of expression of the genomic marker (negative selection).

In another aspect, the method comprises introducing a first polynucleotide comprising, in a direction from 5′ to 3′, a first flanking sequence, a first region comprising a first target sequence, a second region comprising a transcription regulation site, a third region comprising a marker encoding region, and a second flanking sequence. The first flanking sequence and the second flanking sequence are homologous with two or more consecutive nucleotides in the direction of a 5′-end of an insertion site of the genome of the cell, or two or more consecutive nucleotides in the direction of a 3′-end of an insertion site of the genome of the cell. By inserting the first polynucleotide into the cell, the first, second, and third regions of the first polynucleotide are inserted into the genome of the cell to provide first, second, and third regions of the genome. Recombinant cells can be selected on the basis of expression of the marker encoded by the first polynucleotide (positive selection).

Additional aspects of the method will be apparent by the detailed description and drawings provided herein.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram of DNA fragments useful for positive selection and negative selection;

FIG. 2 is a schematic diagram of a method of performing sequential steps of positive selection and a negative selection to select a cell in which a genes are inserted into the genome using a cell with a genome encoding a marker prior to the introduction of a first polynucleotide;

FIG. 3 is a schematic diagram of a method of performing sequential steps of positive selection and negative selection to select a cell into which genes are inserted into the genome using a cell with a genome that does not encode a marker prior to the introduction of a first polynucleotide;

FIG. 4 is a gel electrophoresis separation showing PCR results verifying successive insertion of target genes, which are pylT,S; pylB; and pylC,D, to an E. coli genome; and

FIG. 5 is a gel electrophoresis separation showing PCR results verifying successive insertion of target genes, which are 025B, 4hbd, and cat2, to an E. coli genome.

FIG. 6 is a schematic diagram of a method of successively introducing a gene to a genome by dividing the gene into two pieces.

FIG. 7 is a graph showing the genome introduction efficiency (%) according to the number of selection (solid line: introduction in the size of a gene, dotted line: introduction of small fragments).

FIG. 8A and 8B respectively show the PCR results of verifying successive insertion of sucD-1 and sucD-2, which are the fragments produced by dividing a target sucD into two pieces, to a genome.

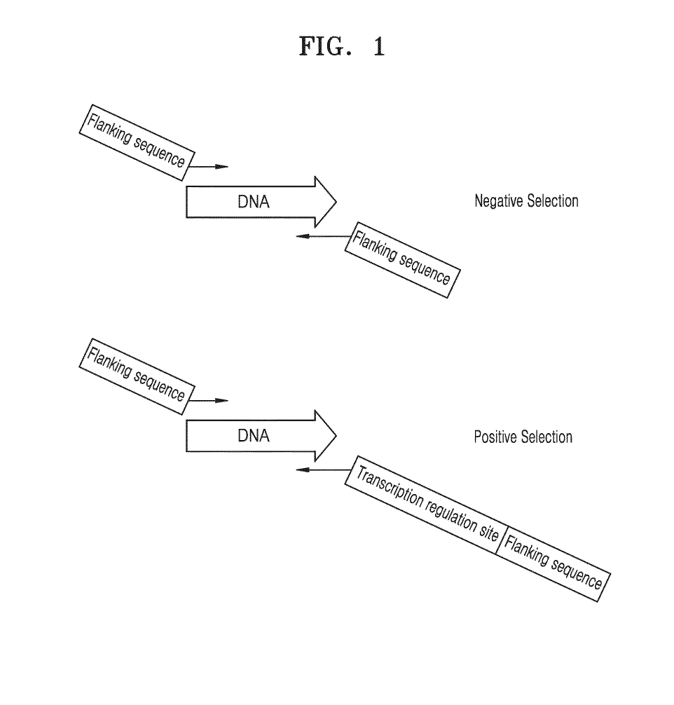

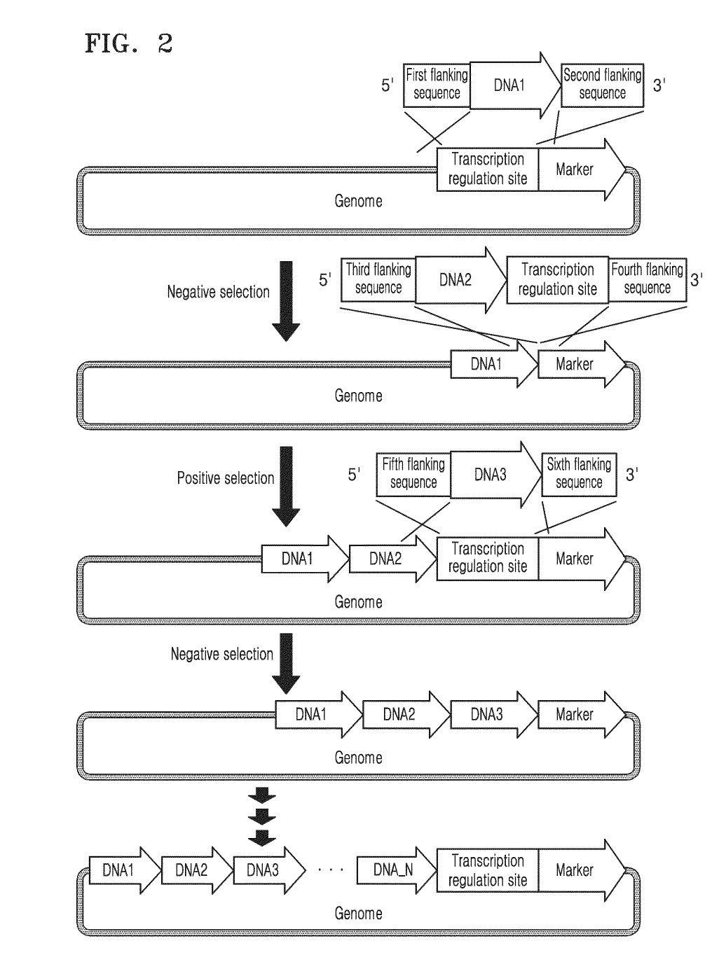

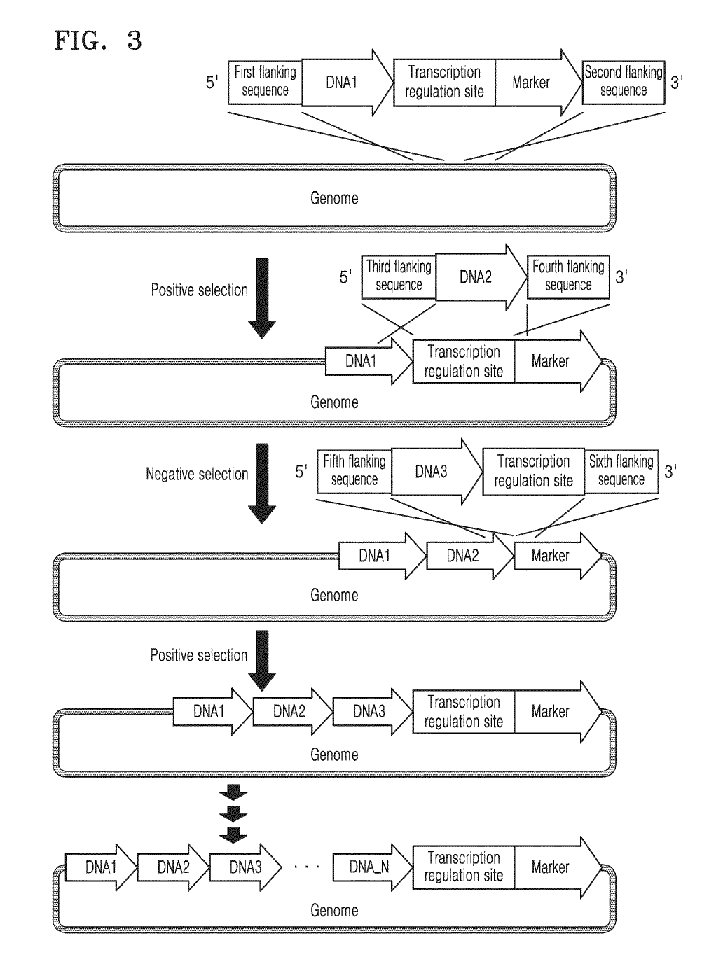

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present disclosure.

Provided is a method of inserting a target sequence into a genome of a cell, when a marker exists in the genome of the cell. The method may include introducing to a cell a first polynucleotide including, in a direction from 5′ to 3′, a first flanking sequence, a first region including a first target sequence, and a second flanking sequence, wherein the genome of the cell comprises, in a direction from 5′ to 3′, a second region including a transcription regulation site of a marker encoding region, and a third region including a marker encoding region, wherein the first flanking sequence is homologous with at least two consecutive polynucleotides in the second region of the genome of the cell, at least two consecutive polynucleotides in a 5′ direction from a 5′-end of the second region, or a combination thereof, and wherein the second flanking sequence is homologous with at least two consecutive polynucleotides in the second region of the genome of the cell, at least two consecutive polynucleotides in a 3′ direction from a 5′-end of the third region, or a combination thereof.

The method includes introducing to a cell a first polynucleotide including, in a direction from 5′ to 3′, a first flanking sequence, a first region including a first target sequence, and a second flanking sequence.

The genome of the cell may include, in a direction from 5′ to 3′, a second region including a transcription regulation site of a marker encoding region, and a third region including a marker encoding region

The cell may be a host cell to which a polynucleotide is to be inserted. For example, the cell may be E. coli, yeast, a fungus, an animal cell, or a plant cell.

The term “genome” used herein refers to all genetic information of a living organism. A genome may consist of DNA (e.g., chromosomes) or RNA, and include a cellular nuclear genome, a mitochondrial genome, or a chloroplast genome.

The genome of the cell may include, in a direction from 5′ to 3′, a second region including a transcription regulation site of a marker encoding region, and a third region including a marker encoding region. For example, a transcription regulation site and a marker may exist within a cell.

The term “transcription regulation site” used herein refers to a nucleic acid segment which may increase or decrease a gene expression of a cell, and may be used interchangeably with the term “regulatory sequence.” A transcription regulation site may be a promoter, an enhancer, an insulator, a silencer, a polynucleotide encoding a transcription factor, or a combination thereof. A promoter, which is a region of DNA that initiates transcription of a gene, is located in the front (upstream) of the gene. A promoter may be a Pribnow box, a TATA box, a B recognition element (BRE), a CAAT box, a response element, or a combination thereof. An enhancer refers to a DNA sequence to which a protein factor increasing a gene transcription level is bound. An enhancer may include an enhancer box (E-box) or a response element. An insulator refers to a genetic boundary element which blocks the interaction between an enhancer and a promoter. A silencer refers to a DNA sequence to which a protein factor decreasing a gene transcription level is bound. A transcription regulation site may preexist in a cell or be inserted (e.g., exogenous transcription regulation site). The length of a transcription regulation site may be from about 5 nucleotides (nt) to about 1000 nt, from about 10 nt to about 800 nt, from about 15 nt to about 600 nt, from about 20 nt to about 400 nt, or from about 25 nt to about 200 nt.

The marker may be a gene which allows positive selection, negative selection, or a combination thereof. For example, a marker may allow both positive selection and negative selection. Positive selection means that, for example, when a gene is inserted to a cell and a transformed cell is selected, the cell is selected under the conditions where a cell expressing a product of the gene may survive. Positive selection can also refer to selection of a cell on the basis of expression of a phenotype or marker. Negative selection means that, for example, when a gene is inserted to a cell and a transformed cell is selected, the cell is selected under the conditions where a cell expressing a product of the gene dies. Negative selection can also refer to selection of a cell on the basis of a lack of expression of a phenotype or marker.

The marker may be a membrane protein, a glycolysis metabolism-related protein, a DNA biosynthesis-related protein, or a combination thereof. The glycolysis metabolism-related protein may be a metabolite or a protein required for glycolysis metabolism. The DNA biosynthesis-related protein may be a protein required for producing DNA. The membrane protein may be a TolC outer membrane channel (TolC) protein. Since transport of a specific material is determined by whether the TolC protein is expressed or not, whether a target protein is inserted may be easily determined by examining whether the TolC protein is expressed or not. For example, in a case where the TolC protein is used as a marker, when the TolC protein is expressed, the cell may survive in a medium including sodium dodecyl sulfate (SDS), since a toxic substance is discharged through the membrane protein. In addition, when the TolC protein is expressed, the cell may not survive when Colicin E1 is introduced to the cell. For example, in positive selection, the cell is selected in a medium including SDS, and the cell where the TolC protein is expressed may survive in the medium including SDS. For example, in negative selection, the cell is selected in a medium including Colicin E1, and the cell where the TolC protein is not expressed may survive in the medium including Colicin E1. The glycolysis metabolism-related protein may be a galactokinase (GalK) protein a component of maltose ABC transporter (MalK) protein. When a galK gene or a malK gene is expressed in a cell, the cell degrades galactose or maltose to produce an acid, which decreases the pH to 6.8 or lower. Then, red colonies are produced to allow positive selection. On the other hand, a cell without a galK gene or a malK gene degrades peptone instead of lactose to produce ammonia, which increases the pH of the medium. Then, white colonies are produced to allow negative selection. The DNA biosynthesis-related protein may be a Thymidylate synthase (ThyA) protein. For example, when ThyA is used as a marker, in the positive selection of the ThyA marker, cells may be screened on the basis of whether a cell survives in an environment without thymine in an M9 minimal medium. The negative selection is performed in an environment where thymine and trimethoprim are included. Trimethoprim blocks conversion of dihydrofolate, which is a byproduct of dTMP synthesis by dUMP methylation, to tetrahydrofolate. Due to the exhaustion of tetrahydrofolate, which is much used in cellular metabolism, a cell including ThyA dies to allow negative selection.

The first flanking sequence may be homologous with at least two consecutive polynucleotides in the second region of the genome of the cell, at least two consecutive polynucleotides in a 5′ direction from a 5′-end of the second region, or a combination thereof.

The second flanking sequence may be homologous with at least two consecutive polynucleotides in the second region of the genome of the cell, at least two consecutive polynucleotides in a 3′ direction from a 5′-end of the third region, or a combination thereof.

The term “flanking sequence” used herein refers to a DNA region which flanks a 5′-end or a 3′-end of a gene or a DNA fragment. The length of a flanking sequence may be from about 2 nt to about 500 nt, from about 2 nt to about 450 nt, from about 2 nt to about 400 nt, from about 2 nt to about 350 nt, from about 2 nt to about 300 nt, or from about 2 nt to about 200 nt. The term “homologous” used herein refers to, for example, similarity or identity between nucleic acid sequences. For example, a polynucleotide sequence homologous with another polynucleotide sequence may be a polynucleotide sequence which is identical or complementary to the other polynucleotide sequence (e.g., sharing 80% or more, 90% or more, 95% or more, or 99% or more, such as 100%, sequence identity). A flanking sequence may be homologous in a 5′-end or a 3′-end direction from an insertion site with a polynucleotide sequence having a length from about 2 nt to about 500 nt, from about 2 nt to about 450 nt, from about 2 nt to about 400 nt, from about 2 nt to about 350 nt, from about 2 nt to about 300 nt, or from about 2 nt to about 200 nt. Typically, the flanking sequence and the corresponding homologous sequence of the genome will have a minimum length of about 5 nt or more, such as about 10 nt or more, 20 nt or more, 30 nt or more, 40 nt or more, or 50 nt or more.

A target gene refers to a gene which is to be inserted into a genome of a cell. An expression product of a target cell is a target protein.

Inserting the polynucleotide into a genome of a cell may be performed by homologous recombination. The term “homologous recombination” used herein refers genetic recombination in which a polynucleotide is exchanged between two similar or identical polynucleotide sequences. Sequences involved in homologous recombination may be identical with each other by 80% or more, 90% or more, 95% or more, or 99% or more.

The first polynucleotide may be inserted into the genome of a cell by homologous recombination, whereby the second region of the genome (transcription regulation site of the marker) is effectively replaced by the first region (target DNA) of the first polynucleotide. The genome then includes a first region (from the first polynucleotide) and a third region in a 3′ direction from 5′-end of the first region.

Insertion of the polynucleotide into the genome, thus, disrupts expression of the marker. The method may, therefore, further include selecting a cell in which the marker is not expressed to perform a negative selection of a cell in which a first target sequence is inserted to a genome of a cell.

The method may further include introducing, after introducing a first polynucleotide to a cell, a second polynucleotide including, in a direction from 5′ to 3′, a third flanking sequence, a fourth region including a second target sequence; a fifth region including a transcription regulatory region; and a fourth flanking sequence, wherein the third flanking sequence is homologous with at least two consecutive polynucleotides in a 5′ direction from a 3′ end of the first region of the genome of the cell; and

the fourth flanking sequence is homologous with at least two consecutive polynucleotides in a 3′ direction from a 5′-end of the third region of the genome of the cell.

The second polynucleotide may be inserted into the genome of the cell, wherein fourth and fifth regions of the polynucleotide provide fourth and fifth regions of the genome. The genome then includes a first region, a fourth region, a fifth region, and a third region in a 3′ direction from 5′-end of the first region.

Introduction of the fourth and fifth regions of the polynucleotide into the genome re-establishes expression of the genomic marker. Thus, the method may further include selecting a cell in which the marker is expressed to perform a positive selection of a cell in which a second target sequence is inserted to a genome of a cell.

The method may further include introducing, after introducing a second polynucleotide to a cell, a third polynucleotide including, in a direction from 5′ to 3′, a fifth flanking sequence, a sixth region including a third target sequence, and a sixth flanking sequence, wherein the fifth flanking sequence is homologous with at least two consecutive polynucleotides in a 5′ direction from 3′-end of the fourth region of the genome of the cell, at least two consecutive polynucleotides in the fifth region, or a combination thereof; and wherein the sixth flanking sequence is homologous with at least two consecutive polynucleotides in a 3′ direction from 5′-end of the third region of the genome of the cell, at least two consecutive polynucleotides in the fifth region, or a combination thereof.

The third polynucleotide may be inserted into the genome of a cell to provide a genome with a sixth region. The genome then includes a first region, a fourth region, a sixth region, and a third region in a 3′ direction from 5′-end of the first region.

Introduction of the sixth region again disrupts expression of the genomic marker. Thus, the method may further include selecting a cell in which the marker is not expressed to perform a negative selection of a cell in which a third target sequence is inserted to a genome of a cell.

The method may further include, after introducing a third polynucleotide into the cell, introducing a fourth polynucleotide into the cell, wherein the fourth polynucleotide includes, in a direction from 5′ to 3′, a seventh flanking sequence, a seventh region including a region of 5′ direction from 3′-end of a fourth target sequence, and an eighth region including a transcription regulatory region, and an eighth flanking sequence,

wherein the seventh flanking sequence is homologous with at least two consecutive nucleotides in a 5′ direction from 3′-end of the six region of the genome of the cell; and

wherein the eighth flanking sequence is homologous with at least two consecutive nucleotides in a 3′ direction from 5′-end of the third region of the genome of the cell,

whereby the seventh and eighth regions of the fourth polynucleotide is introduced into the genome to provide seventh and eighth regions of the genome.

The method may further include, after introducing a fourth polynucleotide into the cell, introducing a fifth polynucleotide into the cell, wherein the fifth polynucleotide includes, in a direction from 5′ to 3′, a ninth flanking sequence, a ninth region including a region of 3′ direction from 5′-end of a fourth target sequence, and a tenth flanking sequence,

wherein the fourth target sequence includes a 3′-end region of the fourth target sequence and a 5′-end region of the fourth target sequence,

wherein the ninth flanking sequence is homologous with at least two consecutive nucleotides in a 5′ direction from 3′-end of the seventh region of the genome of the cell, at least two consecutive nucleotides in the eighth region, or a combination thereof; and

wherein the tenth flanking sequence is homologous with at least two consecutive polynucleotides in a 3′ direction from 5′-end of the third region of the genome of the cell, at least two consecutive nucleotides in the eighth region, or a combination thereof,

whereby the ninth region of the fourth polynucleotide is introduced into the genome to provide a ninth region of the genome.

The fourth polynucleotide and the fifth polynucleotide may be inserted to produce a genome of a cell. The genome then includes a first region, a fourth region, a sixth region, a seventh region, a ninth region, and a third region in a 3′ direction from 5′-end of the first region.

The method may further include selecting a cell in which the marker is expressed to perform a positive selection of a cell in which a 3′-terminal region of the fourth target sequence is inserted to a genome of a cell. The method may further include selecting a cell in which the marker is not expressed to perform a negative selection of a cell in which a 5′-terminal region of a fourth target sequence is inserted to a genome of a cell. Positive selection and negative selection are described above.

According to one embodiment of the present disclosure, when a transcription factor and a marker exist in a cell, target genes may be successively inserted to a genome of a cell. A negative selection may be performed at an odd-numbered insertion, and a positive selection may be alternately performed at an even-numbered insertion to successively verify insertion of two or more target genes. In addition, to increase the efficiency of inserting a target gene, the size of an introduced gene may be decreased before introducing the gene into a genome. For example, fragments of a target gene may be introduced to a genome one by one.

One aspect of the present disclosure provides a method of inserting a target sequence into a genome of a cell when a marker is not included in a cell genome. The method can comprise introducing into the cell a first polynucleotide including, in a direction from 5′ to 3′, a first flanking sequence, a first region including a first target sequence, a second region including a transcription regulatory region, a third region including a marker encoding region, and a second flanking sequence, wherein the first flanking sequence and the second flanking sequence are homologous with two or more consecutive nucleotides in the direction of a 5′-end of an insertion site of the genome of the cell, or two or more consecutive nucleotides in the direction of a 3′-end of an insertion site of the genome of the cell. The flanking sequence, the target sequence, the transcription regulation site, the marker encoding region, and the insertion to a cell or to a genome of a cell are described above. The insertion site refers to a site to which a polynucleotide sequence is inserted.

The first polynucleotide may be inserted into the genome of the cell, wherein the first and second regions of the first polynucleotide provide first and second regions of the genome. The genome then includes a first region, a second region, and a third region in a 3′ direction from 5′-end of the first region.

Introduction of the first polynucleotide into the genome establishes expression of a marker. Thus, the method may further include selecting a cell in which the marker is expressed to perform a positive selection of a cell in which a first target sequence is inserted to a genome of a cell.

The method may further include introducing, after introducing a first polynucleotide to a cell, a second polynucleotide including, in a direction from 5′ to 3′, a third flanking sequence, a fourth region including a second target sequence, and a fourth flanking sequence, wherein the third flanking sequence is homologous with at least two consecutive polynucleotides in a 5′ direction from 3′-end of the first region of the genome of the cell, at least two consecutive polynucleotides in the second region, or a combination thereof, and wherein the fourth flanking sequence is homologous with at least two consecutive polynucleotides in the second region of the genome of the cell, at least two consecutive polynucleotides in a 3′ direction from 5′-end of the third region, or a combination thereof.

The second polynucleotide may be inserted into the genome of the cell, wherein the fourth region of the second polynucleotide provides a fourth region in the genome. The genome then includes a first region, a fourth region, a third region in a 3′ direction from 5′-end of the first region.

Introduction of the second polynucleotide into the genome disrupts expression of the marker. Thus, The method may further include selecting a cell in which the marker is not expressed to perform a negative selection of a cell in which a second target sequence is inserted to a genome of a cell.

The method may further include introducing, after introducing a second polynucleotide to a cell, a third polynucleotide including, in a direction from 5′ to 3′, a fifth flanking sequence, a sixth region including a third target sequence; a fifth region including a transcription regulatory region; and a sixth flanking sequence, wherein the fifth flanking sequence is homologous with at least two consecutive polynucleotides in a 5′ direction from a 3′-end of the fourth region of the genome of the cell; and wherein the sixth flanking sequence is homologous with at least two consecutive polynucleotides in a 3′ direction from a 5′-end of the third region of the genome of the cell.

The third polynucleotide may be inserted into the genome of the cell, wherein the fifth and sixth regions of the third polynucleotide provide fifth and sixth regions in the genome. The genome then includes a first region, a fourth region, a sixth region, a fifth region, and a third region in a 3′ direction from 5′-end of the first region.

Introduction of the third polynucleotide re-establishes expression of the marker. Thus, the method may further include selecting a cell in which the marker is expressed to perform a positive selection of a cell in which a third target sequence is inserted to a genome of a cell.

The method may further include, after introducing a third polynucleotide into the cell, introducing a fourth polynucleotide into the cell, wherein the fourth polynucleotide includes, in a direction from 5′ to 3′, a seventh flanking sequence, a seventh region including a region of 5′ direction from 3′-end of a fourth target sequence, and an eighth flanking sequence,

wherein the seventh flanking sequence is homologous with at least two consecutive nucleotides in a 5′ direction from 3′-end of the sixth region of the genome of the cell, at least two consecutive nucleotides in the fifth region, or a combination thereof; and

wherein the eighth flanking sequence is homologous with at least two consecutive nucleotides in a 3′ direction from 5′-end of the third region of the genome of the cell, at least two consecutive nucleotides in the fifth region, or a combination thereof,

whereby the seventh region of the fourth polynucleotide is inserted into the genome of the cell to provide a seventh region of the genome.

The method may further include, after introducing a fourth polynucleotide into the cell, introducing a fifth polynucleotide into the cell, wherein the fifth polynucleotide includes, in a direction from 5′ to 3′, a ninth flanking sequence, an eighth region including a region of 3′ direction from 5′-end of a fourth target sequence, a ninth region including a transcription regulatory region, and a tenth flanking sequence,

wherein the fourth target sequence comprises a 3′-end region of the fourth target sequence and a 5′-end region of the fourth target sequence,

wherein the ninth flanking sequence is homologous with at least two consecutive nucleotides in a 5′ direction from 3′-end of the seventh region of the genome of the cell; and

wherein the tenth flanking sequence is homologous with at least two consecutive nucleotides in a 3′ direction from 5′-end of the third region of the genome of the cell,

whereby the eighth and ninth regions of the fourth polynucleotide is introduced into the genome to provide eighth and ninth regions of the genome.

The fourth polynucleotide and the fifth polynucleotide may be inserted to produce a genome of a cell, wherein the genome includes a first region, a fourth region, a sixth region, a seventh region, an eighth region, a ninth region, and a third region in a 3′ direction from 5′-end of the first region.

The method may further include selecting a cell in which the marker is not expressed to perform a negative selection of a cell in which a 3′-terminal region of a fourth target sequence is inserted to a genome of a cell. The method may further include selecting a cell in which the marker is expressed to perform a positive selection of a cell in which a 5′-terminal region of the fourth target sequence is inserted to a genome of a cell. Negative selection and positive selection are described above.

According to one embodiment of the present disclosure, when a transcription factor and a marker are introduced from the outside of a cell to the inside of a cell, target genes may be successively inserted to a genome of a cell. A positive selection may be performed at an odd-numbered insertion, and a negative selection may be alternately performed at an even-numbered insertion to successively verify insertion of two or more target genes. In addition, to increase the efficiency of inserting a target gene, the size of an introduced gene may be decreased before introducing the gene into a genome. For example, fragments of a target gene may be introduced to a genome one by one.

The methods described herein refer to the use of first, second, and third polynucleotides and accompanying positive or negative selection steps. However, the method is not limited thereto. The introduction of additional polypeptides followed by alternating positive or negative selection steps can be employed.

Hereinafter, the present disclosure will be described in further detail with reference to examples. It will be obvious to a person having ordinary skill in the art that these examples are illustrative purposes only and are not to be construed to limit the scope of the present disclosure.

### EXAMPLE 1

### Using Intracellular Gene (tolC) as Marker to Insert Pyrrolysine Gene Cluster to Genome

### 1.1 Selection of Pyrrolysine Gene Cluster as Target Gene

Pyrrolysine is an amino acid represented by Chemical Formula 1:

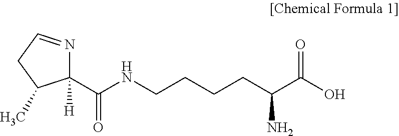 [SMILES: C[C@@H]1CC=N[C@H]1C(=O)NCCCC[C@H](N)C(=O)O]

To prepare a protein containing pyrrolysine, the five genes pylT, pylS, pylB, pylC, and pylD genes were used as a pyrrolysine gene cluster and successively inserted into the genome of E. coli. The pylT gene is a gene encoding a tRNA which inserts pyrrolysine to a TAG codon, the pylS gene is a gene synthesizing pyrrolysyl-tRNA synthetase which links pyrrolysine with a tRNA, and pylB, pylC, and pylD genes are genes that are needed to synthesize L-pyrrolysine from L-lysine. A pylT,S gene, which was formed by connecting pylT and pylS, was selected as a first target gene, pylB was selected as a second target gene, and pylC,D, which was formed by connecting pylC and pylD, was selected as a third target gene.

### 1.2 Preparation of DNA Fragment for Insertion to Genome

A tolC gene included in E. coli genome was used as a marker, and a DNA fragment for negative selection or positive selection was prepared.

A DNA fragment including, in a 3′ direction from 5′-end, a first flanking sequence (SEQ ID NO: 1), a polynucleotide that was the same as the pylT,S gene which was a first target gene (SEQ ID NO: 2), and a second flanking sequence (SEQ ID NO: 3) was prepared. The first flanking sequence (SEQ ID NO: 1) was a polynucleotide that was the same as 50 nucleotides located in a 3′ direction from 5′-end on a transcription regulation site of the tolC gene which was to be inserted to an E. coli genome. The second flanking sequence (SEQ ID NO: 3) was a polynucleotide that was the same as 50 nucleotides located in a 3′ direction from 5′-end of the tolC gene which was to be inserted to an E. coli genome. The first flanking sequence and the second flanking sequence were designed to form a homology arm for homologous recombination to delete a transcription regulation site of the tolC gene of an E. coli genome and insert the first target gene, which was pylT,S, to the site of the transcription regulation site of the tolC gene. When the first target gene, which was pylT,S gene was inserted, the pylT,S, and the tolC gene were arranged in a 3′ direction from 5′-end.

Next, as a DNA fragment for positive selection, a DNA fragment including, in a 3′ direction from 5′-end, a third flanking sequence (SEQ ID NO: 4), a polynucleotide that was the same as the pylB gene which was a second target gene (SEQ ID NO: 5), a transcription regulation site (SEQ ID NO: 6), and a fourth flanking sequence (SEQ ID NO: 7) was prepared. The third flanking sequence (SEQ ID NO: 4) was a polynucleotide that was the same as 50 nucleotides located in a 5′ direction from 3′-end of the pylT,S gene, which was the first target gene. The transcription regulation site (SEQ ID NO: 6) was a polynucleotide including a T7 promoter, an untranslated region (UTR), and a ribosome binding site. The fourth flanking sequence (SEQ ID NO: 7) was a polynucleotide that was the same as 50 nucleotides located in a 3′ direction from 5′-end of the tolC gene. The third flanking sequence and the fourth flanking sequence were designed to form a homology arm for homologous recombination to insert the second target gene, which was pylB, and the transcription regulation site between the pylT,S gene, which was the first target gene, and the tolC gene in a 3′ direction from 5′-end. When the second target gene, which was pylB, and the transcription regulation site were inserted, the first target gene, which was pylT,S; the second target gene, which was pylB; the transcription regulation site; and the tolC gene were arranged in a 3′ direction from 5′-end.

Next, as a DNA fragment for negative selection, a DNA fragment including, in a 3′ direction from 5′-end, a fifth flanking sequence (SEQ ID NO: 8), a polynucleotide that was the same as the pylC,D gene which was a third target gene (SEQ ID NO: 9), and a sixth flanking sequence (SEQ ID NO: 10) was prepared. The fifth flanking sequence (SEQ ID NO: 8) was a polynucleotide that was the same as 50 nucleotides located in a 5′ direction from 3′-end of the inserted pylB gene, which was the second target gene. The sixth flanking sequence (SEQ ID NO: 10) was a polynucleotide that was the same as 50 nucleotides located in a 3′ direction from 5′-end of the tolC gene. The fifth flanking sequence and the sixth flanking sequence were designed to form a homology arm for homologous recombination to delete the transcription regulation site and insert the third target gene, which was pylC,D, to the transcription regulation site. When the third target gene, which was pylC,D, was inserted, the first target gene, which was pylT,S, the second target gene, which was pylB, the third target gene, which was pylC,D, and the tolC gene were arranged in a 3′ direction from 5′-end.

### 1.3 Insertion of DNA Fragment Including First Target Gene to E. coli, and Negative Selection to Verify Insertion

The DNA fragment prepared in Example 1.2, wherein the DNA fragments included, in a direction from 5′ to 3′, a first flanking sequence (SEQ ID NO: 1), a polynucleotide encoding pylT,S (SEQ ID NO: 2), and a second flanking sequence (SEQ ID NO: 3), was inserted into E. coli. The insertion was performed by electroporation.

Then, the E. coli was cultured in an LB medium. Specifically, the culturing was performed as described below. An E. coli strain that was to be engineered was inoculated to 3 ml of an LB medium. Since a DNA fragment is inserted to a genome by lambda red recombination, an E. coli strain, which included a gene encoding lambda red proteins, was used.

To prevent expression of lambda red protein coding genes to reduce background mutation, the culturing was performed at 30° C. until the optical density (OD) value at 600 nanometers reached 0.6. Then, the culturing was performed at 42° C. for 15 minutes. In the E. coli strain used in the present experiment, the genes encoding lambda red proteins were controlled by a pL promoter. To activate the pL promoter, the culturing was performed at 42° C. to remove a repressor attached to the promoter.

After centrifugation of 1 ml of the E. coli culture solution at 4° C. and at 13,000 rpm for 1 minute, the supernatant was removed. One ml of distilled water was added to a pellet to wash the cells, the resulting solution was centrifuged at 4° C. and at 13,000 rpm for 1 minute. To increase the efficiency of electroporation, the washing was performed once again to remove salts. After removing the supernatant, 50 ul (microliters) of the resulting solution including the prepared DNA fragment (including about 1,000 ng of DNA) was added to the culture medium to mix with the cells. After performing electroporation with the mixed cells, the E. coli was transported to 3 ml of an LB medium and the resulting solution was cultured at 30° C. for 3 hours.

To perform a negative selection with the cultured E. coli, the cultured E. coli was successively diluted, smeared on a solid medium including 0.1% (v/v) Colicin E1, and then cultured overnight. Since Colicin E1 is introduced to a cell and shows cellular toxicity when the marker is expressed, a cell where the marker is expressed dies in a culture medium including Colicin E1. Therefore, a negative selection may be performed.

### 1.4 Insertion of DNA Fragment Including Second Target Gene to E. coli, and Positive Selection to Verify Insertion

The E. coli, to which the first target gene was inserted, was selected by negative selection. The DNA fragment prepared in Example 1.2, wherein the DNA fragment included, in a direction from 5′ to 3′, a third flanking sequence (SEQ ID NO: 4), a polynucleotide encoding pylB (SEQ ID NO: 5), a transcription regulation site (SEQ ID NO: 6), and a fourth flanking sequence (SEQ ID NO: 7), was inserted into the genome of E. coli by the method described in Example 1.3, and then the resulting E. coli strain was cultured.

To perform a positive selection with the cultured E. coli, the cultured E. coli was successively diluted, smeared on a solid medium including 0.01% (v/v) SDS, and then cultured overnight. When the marker is expressed, the SDS introduced to the cell is discharged by the TolC protein to the outside of the cell, and thus the cell may survive in a medium including SDS. Therefore, a positive selection may be performed.

### 1.5 Insertion of DNA Fragment Including Third Target Gene to E. coli, and Negative Selection to Verify Insertion

The E. coli, to which the first target gene was inserted, was selected by negative selection. The DNA fragment prepared in Example 1.2, wherein the DNA fragment included, in a direction from 5′ to 3′, a fifth flanking sequence (SEQ ID NO: 8), a polynucleotide encoding pylC,D (SEQ ID NO: 9), and a sixth flanking sequence (SEQ ID NO: 10), was inserted to E. coli by the method described in Example 1.3, and then the resulting E. coli strain was cultured.

To perform a negative selection with the cultured E. coli, the cultured E. coli was successively diluted, smeared on a solid medium including 0.1% (v/v) Colicin E1, and then cultured overnight. Since Colicin E1 is introduced to a cell and shows cellular toxicity when the marker is not expressed, a cell where the marker is not expressed dies in a culture medium including Colicin E1. Therefore, a negative selection may be performed.

### 1.6 Verification of Target Gene Inserted to E. coli Genome

The E. coli, to which the first target gene, the second target gene, and the third target gene were inserted in Examples 1.3 to 1.5, was obtained through positive selection or negative selection. To verify the insertion of the first target gene, the second target gene, and the third target gene to the E. coli genome, a polymerase chain reaction (PCR) was performed.

Specifically, an E. coli colony was selected from a solid medium cultured overnight, and a colony PCR was performed by using the E. coli colony.

To verify the insertion of the first target gene, a first forward primer (SEQ ID NO: 11) and a second reverse primer (SEQ ID NO: 12) which may specifically amplify the pylT,S gene, which was the first target gene, were used.

To verify the insertion of the second target gene, a third forward primer (SEQ ID NO: 13) and a fourth reverse primer (SEQ ID NO: 14) which may specifically amplify the pylB gene, which was the second target gene, were used.

To verify the insertion of the third target gene, a fifth forward primer (SEQ ID NO: 15) and a sixth reverse primer (SEQ ID NO: 16) which may specifically amplify the pylC,D gene, which was the third target gene, were used.

Table 1 shows the composition of reactants for the PCR.

TABLE 1

| 2X Taq | | Forward primer | Reverse primer | Template (picked |
|---|---|---|---|---|
| premix | Water | (10 μM) | (10 μM) | E. coli colony) |
| 10 ul | 7 ul | 1 ul | 1 ul | 1 ul |

The PCR was performed by using S-100 Thermal Cycler (Biorad) under the PCR conditions shown in Table 2.

TABLE 2

| | | Temperature | Time | |
|---|---|---|---|---|
| Initial denaturation | | 95° C. | 3 | min |
| 35 cycles | Denaturation | 95° C. | 30 | sec |
| | Annealing | 60° C. | 30 | sec |
| | Extension | 72° C. | 1 min 30 sec | |
| Final extension | | 72° C. | 10 | min |

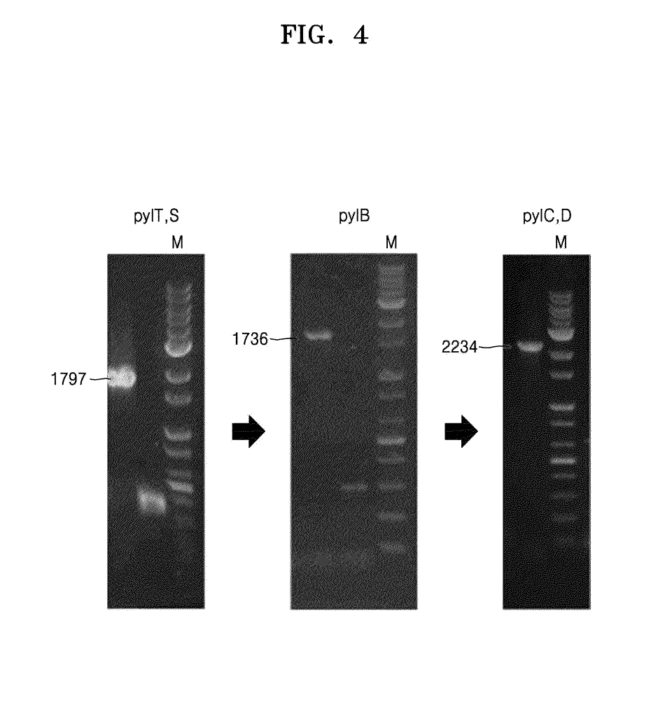

The product obtained by performing a PCR under the conditions shown above underwent electrophoresis in an agarose gel to verify the size of the DNA fragment inserted to the E. coli genome. The result is shown in FIG. 4. As shown in FIG. 4, it was verified that the first target gene, the second target gene, and the third target gene were inserted to the E. coli genome. In addition, the sequence of a DNA fragment was analyzed with respect to E. coli showing a predicted band size to finally verify the insertion of the DNA fragment. The result showed that all the target gene sequences were inserted to the E. coli selected by positive selection and negative selection.

### EXAMPLE 2

### Insertion of 1,4-Butanediol Gene Cluster to Genome by Using Intracellular Gene (tolC) as Marker

### 2.1 Selection of 1,4-Butanediol Gene Cluster as Target Gene

To produce 1,4-butanediol (BDO) in E. coli, 025B (aldehyde dehydrogenase gene), 4HBd (4HB dehydrogenase), and cat2 (4-hydroxybutyryl-CoA transferase) genes, which are not included in E. coli, were successively inserted to E. coli. The 025B was selected as a first target gene, the 4HBd gene was selected as a second target gene, and the cat2 gene was selected as a third target gene.

### 2.2 Preparation of DNA Fragment for Insertion to Genome

The tolC gene existing in E. coli was used as a marker, and a DNA fragment for negative selection or positive selection was prepared.

A DNA fragment including, in a 3′ direction from 5′-end, a seventh flanking sequence (SEQ ID NO: 17), a polynucleotide that was the same as the 025B gene which was a first target gene (SEQ ID NO: 18), and an eighth flanking sequence (SEQ ID NO: 19) was prepared. The seventh flanking sequence (SEQ ID NO: 17) was a polynucleotide that was the same as 300 nucleotides located in a 3′ direction from 5′-end of a transcription regulation site of the tolC gene which was to be inserted to an E. coli genome. The eighth flanking sequence (SEQ ID NO: 19) was a polynucleotide that was the same as 300 nucleotides located in a 3′ direction from 5′-end of the tolC gene which was to be inserted to an E. coli genome. The seventh flanking sequence and the eighth flanking sequence were designed to form a homology arm for homologous recombination to delete a transcription regulation site of the tolC gene of an E. coli genome and insert the first target gene, which was 025B, to the site of the transcription regulation site of the tolC gene. When the first target gene, which was 025B, was inserted, the 025B gene and the tolC gene were arranged in a 3′ direction from 5′-end.

Next, as a DNA fragment for positive selection, a DNA fragment including, in a 3′ direction from 5′-end, a ninth flanking sequence (SEQ ID NO: 20), a polynucleotide that was the same as the 4HBd gene which was a second target gene (SEQ ID NO: 21), a transcription regulation site (SEQ ID NO: 6), and a tenth flanking sequence (SEQ ID NO: 2) was prepared. The ninth flanking sequence (SEQ ID NO: 20) was a polynucleotide that was the same as 300 nucleotides located in a 5′ direction from 3′-end of the inserted 025B gene, which was the first target gene. The transcription regulation site (SEQ ID NO: 6) was a polynucleotide including a T7 promoter, a UTR, and a ribosome binding site. The tenth flanking sequence (SEQ ID NO: 22) was a polynucleotide that was the same as 300 nucleotides located in a 3′ direction from 5′-end of the tolC gene.

The ninth flanking sequence and the tenth flanking sequence were designed to form a homology arm for homologous recombination to insert the second target gene, which was 4HBd, and the transcription regulation site between the 025B gene, which was the first target gene, and the tolC gene. When the second target gene, which was 4HBd, and the transcription regulation site were inserted, the first target gene, which was 025B; the second target gene, which was 4HBd; the transcription regulation site; and the tolC gene were arranged in a 3′ direction from 5′-end.

Next, as a DNA fragment for negative selection, a DNA fragment including, in a 3′ direction from 5′-end, an eleventh flanking sequence (SEQ ID NO: 23), a polynucleotide the that was same as the cat2 gene which was a third target gene (SEQ ID NO: 24), and a twelfth flanking sequence (SEQ ID NO: 25) was prepared. The eleventh flanking sequence (SEQ ID NO: 23) was a polynucleotide the that was same as 300 nucleotides located in a 5′ direction from 3′-end of the inserted 4HBd gene, which was the second target gene. The twelfth flanking sequence (SEQ ID NO: 25) was a polynucleotide that was the same as 300 nucleotides located in a 3′ direction from 5′-end of the tolC gene. The eleventh flanking sequence and the twelfth flanking sequence were designed to form a homology arm for homologous recombination to delete the transcription regulation site and insert the third target gene, which was cat2, to the site of the transcription regulation site. When the third target gene, which was cat2, was inserted, the first target gene, which was 025B; the second target gene, which was 4HBd; the third target gene, which was cat2; and the tolC gene were arranged in a 3′ direction from 5′-end.

### 2.3 Successive Insertion of DNA Fragment Including First Target Gene, DNA Fragment Including Second Target Gene, and DNA Fragment Including Third Target Gene to E. coli, and Negative or Positive Selection to Verify Insertion

The DNA fragment including the first target gene, the DNA fragment including the second target gene, and the DNA fragment including the third target gene, which were prepared in Example 2.2, were successively inserted to E. coli by the methods described in Example 1.3 to Example 1.5, and the E. coli was selected by negative or positive selection.

### 2.4 Verification Target Gene Inserted to E. coli Genome

The E. coli, to which a first target gene, a second target gene, and a third target gene were inserted in Example 2.3, was obtained through positive selection or negative selection. To verify the insertion of the first target gene, the second target gene, and the third target gene to the E. coli genome, a PCR was performed.

Specifically, an E. coli colony was selected from a solid medium cultured overnight, and a colony PCR was performed by using the colony.

To verify the insertion of the first target gene, a seventh forward primer (SEQ ID NO: 26) and an eighth reverse primer (SEQ ID NO: 27) which may specifically amplify the 025B gene, which was the first target gene, were used.

To verify the insertion of the second target gene, a ninth forward primer (SEQ ID NO: 28) and a tenth reverse primer (SEQ ID NO: 29) which may specifically amplify the 4HBd gene, which was the second target gene, were used.

To verify the insertion of the third target gene, an eleventh forward primer (SEQ ID NO: 30) and a twelfth reverse primer (SEQ ID NO: 31) which may specifically amplify the cat2 gene, which was the third target gene, were used.

Table 3 shows the composition of reactants for the PCR.

TABLE 3

| 2X Taq | | Forward primer | Reverse primer | Template (picked |
|---|---|---|---|---|
| premix | Water | (10 μM) | (10 μM) | E. coli colony) |
| 10 ul | 7 ul | 1 ul | 1 ul | 1 ul |

The PCR was performed by using S-100 Thermal Cycler (Biorad) under the PCR conditions shown in Table 4.

TABLE 4

| | | Temperature | Time | |
|---|---|---|---|---|
| Initial denaturation | | 95° C. | 3 | min |
| 35 cycles | Denaturation | 95° C. | 30 | sec |
| | Annealing | 60° C. | 30 | sec |
| | Extension | 72° C. | 2 min 30 sec | |
| Final extension | | 72° C. | 10 | min |

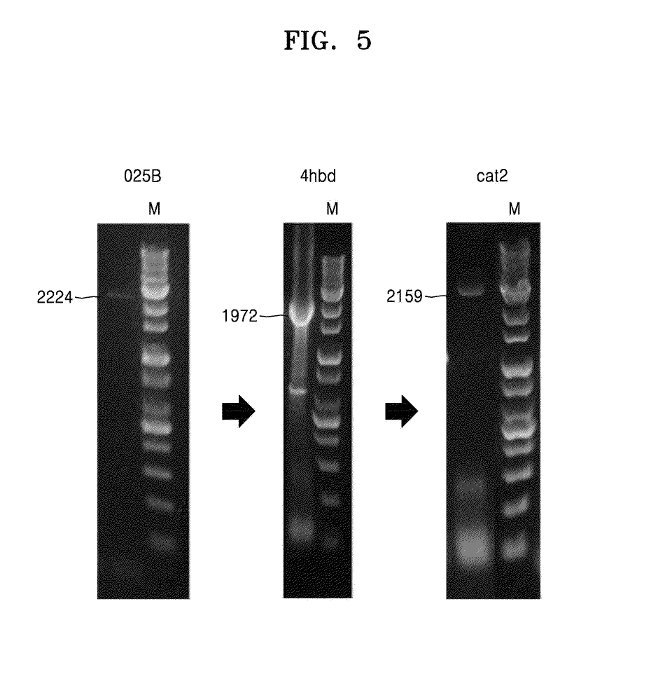

The product obtained by performing a PCR under the conditions shown above underwent electrophoresis in an agarose gel to verify the size of the DNA fragment inserted into the E. coli genome. The result is shown in FIG. 5. As shown in FIG. 5, it was verified that the first target gene, the second target gene, and the third target gene were inserted to the E. coli genome. In addition, the sequence of a DNA fragment was analyzed with respect to E. coli showing a predicted band size to finally verify the insertion of the DNA fragment. The result showed that all the target gene sequences were inserted to the E. coli selected by positive selection and negative selection.

### EXAMPLE 3

### Insertion of Pyrrolysine Gene Cluster to Genome by Using Externally Inserted Marker Gene (tolC)

### 3.1 Preparation of DNA Fragment for Insertion to Genome

A DNA fragment was prepared by the same method as that of Example 1, except that a marker gene was externally inserted. As a gene allowing positive selection or negative selection, a tolC gene, a galK gene, a malK gene, or a thyA gene may be used.

Specifically, a DNA fragment including, in a 3′ direction from 5′-end, a thirteenth flanking sequence (SEQ ID NO: 32); a polynucleotide that was the same as the pylT,S gene which was a first target gene (SEQ ID NO: 2), a transcription regulation site (SEQ ID NO: 6); a marker gene, which was the tolC gene (SEQ ID NO: 33); and the fourteenth flanking sequence (SEQ ID NO: 34) was prepared. The transcription regulation site (SEQ ID NO: 6) was a polynucleotide including a T7 promoter, a UTR, and a ribosome binding site. The thirteenth flanking sequence (SEQ ID NO: 32) was a polynucleotide that was the same as 50 nucleotides of the region to be inserted to an E. coli genome. The fourteenth flanking sequence (SEQ ID NO: 34) was a polynucleotide that was the same as 50 nucleotides of the region to be inserted to an E. coli genome, and a poly nucleotide that was the same as the genome region separated in a 3′ direction from the genome region that was the same as the thirteenth flanking sequence. The thirteenth flanking sequence and the fourteenth flanking sequence were designed to form a homology arm for homologous recombination to insert to an E. coli genome the first target gene, which was pylT,S; the transcription regulation site; and the marker gene, which was the tolC gene. When the first target gene, which was pylT,S, the transcription regulation site, and the marker gene, which was the tolC gene, were inserted to an E. coli genome, the pylT,S gene; the transcription regulation site; and the tolC gene were arranged in a 3′ direction from 5′-end.

Next, as a DNA fragment for negative selection, a DNA fragment including, in a 3′ direction from 5′-end, a third flanking sequence (SEQ ID NO: 4), a polynucleotide that was the same as the pylB gene which was a second target gene (SEQ ID NO: 5), and a fourth flanking sequence (SEQ ID NO: 7) was prepared. The third flanking sequence (SEQ ID NO: 4) was a polynucleotide that was the same as 50 nucleotides located in a 5′ direction from 3′-end of the pylT,S gene, which was the first target gene. The fourth flanking sequence (SEQ ID NO: 7) was a polynucleotide that was the same as 50 nucleotides located in a 3′ direction from 5′-end of the tolC gene. The third flanking sequence and the fourth flanking sequence were designed to form a homology arm for homologous recombination to delete the transcription regulation site and insert the second target gene to the site of the transcription regulation site. When the second target gene, which was pylB, and the transcription regulation site were inserted, the first target gene, which was pylT,S; the second target gene, which was pylB; and the tolC gene were arranged in a 3′ direction from 5′-end.

Next, as a DNA fragment for positive selection, a DNA fragment including, in a 3′ direction from 5′-end, a fifth flanking sequence (SEQ ID NO: 8), a polynucleotide that was the same as the pylC,D gene which was a third target gene (SEQ ID NO: 9), a transcription regulation site (SEQ ID NO: 6), and a sixth flanking sequence (SEQ ID NO: 10) was prepared. The fifth flanking sequence (SEQ ID NO: 8) was a polynucleotide that was the same as 50 nucleotides located in a 5′ direction from 3′-end of the pylB gene, which was the second target gene. The sixth flanking sequence (SEQ ID NO: 10) was a polynucleotide that was the same as 50 nucleotides located in a 3′ direction from 5′-end of the tolC gene. The fifth flanking sequence and the sixth flanking sequence were designed to form a homology arm for homologous recombination to insert the third target gene, which was pylC,D, and the transcription regulation site between the pylB gene, which was the second target gene, and the tolC gene. When the third target gene, which was pylC,D, and the transcription regulation site were inserted, the first target gene, which was pylT,S; the second target gene, which was pylB; the third target gene, which was pylC,D; the transcription regulation site; and the tolC gene were arranged in a 3′ direction from 5′-end.

### 3.2 Successive Insertion of DNA Fragment Including First Target Gene, DNA Fragment Including Second Target Gene, and DNA Fragment Including Third Target Gene to E. coli, and Negative or Positive Selection to Verify Insertion

The DNA fragment including the first target gene, the DNA fragment including the second target gene, and the DNA fragment including the third target gene, which were prepared in Example 2.2, were successively inserted to E. coli by the methods described in Example 1.3 to Example 1.5.

Then, according to the methods described in Example 1.3 to Example 1.5, E. coli, to which the DNA fragment including the first target gene was inserted, was selected by positive selection, E. coli, to which the DNA fragment including the second target gene was inserted, was selected by negative selection, and E. coli, to which the DNA fragment including the third target gene was inserted, was selected by positive selection.

### 3.3 Verification Target Gene Inserted to E. coli Genome

The E. coli, to which a first target gene, a second target gene, and a third target gene were inserted in Example 3.2, was obtained through positive selection or negative selection. To verify the insertion of the first target gene, the second target gene, and the third target gene to the E. coli genome, a PCR was performed.

Specifically, an E. coli colony was selected from a solid medium cultured overnight, and a colony PCR was performed by using the E. coli colony.

To verify the insertion of the first target gene, a first forward primer (SEQ ID NO: 11) and a second reverse primer (SEQ ID NO: 12) which may specifically amplify the pylT,S gene, which was the first target gene, were used.

To verify the insertion of the second target gene, a third forward primer (SEQ ID NO: 13) and a fourth reverse primer (SEQ ID NO: 14) which may specifically amplify the pylB gene, which was the second target gene, were used.

To verify the insertion of the third target gene, a fifth forward primer (SEQ ID NO: 15) and a sixth reverse primer (SEQ ID NO: 16) which may specifically amplify the pylC,D gene, which was the third target gene, were used.

Table 5 shows the composition of reactants for the PCR.

TABLE 5

| 2X Taq | | Forward primer | Reverse primer | Template (picked |
|---|---|---|---|---|
| premix | Water | (10 μM) | (10 μM) | E. coli colony) |
| 10 ul | 7 ul | 1 ul | 1 ul | 1 ul |

The PCR was performed by using an S-100 Thermal Cycler (Biorad) under the PCR conditions shown in Table 6.

TABLE 6

| | | Temperature | Time | |
|---|---|---|---|---|
| Initial denaturation | | 95° C. | 3 | min |
| 35 cycles | Denaturation | 95° C. | 30 | sec |
| | Annealing | 60° C. | 30 | sec |
| | Extension | 72° C. | 1 min 30 sec | |
| Final extension | | 72° C. | 10 | min |

The product obtained by performing a PCR under the conditions shown above underwent electrophoresis in an agarose gel to verify the size of the DNA fragment inserted into the E. coli genome. The result verified that the first target gene, the second target gene, and the third target gene were inserted to the E. coli genome. In addition, the sequence of a DNA fragment was analyzed with respect to E. coli showing a predicted band size to finally verify the insertion of the DNA fragment. The result showed that all the target gene sequences were inserted to the E. coli selected by positive selection and negative selection.

### EXAMPLE 4

### Insertion of 1,4-Butanediol Gene Cluster to Genome by Using Externally Inserted Marker Gene (tolC)

### 4.1 Preparation of DNA Fragment for Insertion to Genome

A DNA fragment was prepared by the same method as that of Example 2, except that a marker gene was externally inserted. As a gene allowing positive selection or negative selection, a tolC gene, a galK gene, a malK gene, or a thyA gene may be used.

Specifically, a DNA fragment including, in a 3′ direction from 5′-end, a thirteenth flanking sequence (SEQ ID NO: 32), a polynucleotide that was the same as the 025B gene which was a first target gene (SEQ ID NO: 18), a transcription regulation site (SEQ ID NO: 6), a marker gene, which was the tolC gene (SEQ ID NO: 32), and the fourteenth flanking sequence (SEQ ID NO: 34) was prepared. The transcription regulation site (SEQ ID NO: 6) was a polynucleotide including a T7 promoter, a UTR, and a ribosome binding site. The thirteenth flanking sequence (SEQ ID NO: 32) was a polynucleotide that was the same as 50 nucleotides of the region to be inserted to an E. coli genome. The fourteenth flanking sequence (SEQ ID NO: 34) was a polynucleotide which was the same as 50 nucleotides of the region to be inserted to an E. coli genome and the same as the genome region separated in a 3′ direction from the genome region that was the same as the thirteenth flanking sequence toward a 3′-end. The thirteenth flanking sequence and the fourteenth flanking sequence were designed to form a homology arm for homologous recombination to insert to an E. coli genome the first target gene, which was 025B; the transcription regulation site; and the marker gene, which was the tolC gene. When the first target gene, which was 025B, the transcription regulation site, and the marker gene, which was the tolC gene, were inserted to an E. coli genome, the 025B gene, the transcription regulation site, and the tolC gene were arranged in a 3′ direction from 5′-end.

Next, as a DNA fragment for negative selection, a DNA fragment including, in a 3′ direction from 5′-end, a third flanking sequence (SEQ ID NO: 4), a polynucleotide that was the same as the 4HBd gene which was a second target gene (SEQ ID NO: 21), and a fourth flanking sequence (SEQ ID NO: 7) was prepared. The third flanking sequence (SEQ ID NO: 4) was a polynucleotide that was the same as 50 nucleotides located in a 5′ direction from 3′-end of the 025B gene, which was the first target gene. The fourth flanking sequence (SEQ ID NO: 7) was a polynucleotide that was the same as 50 nucleotides located in a 3′ direction from 5′-end of the tolC gene. The third flanking sequence and the fourth flanking sequence were designed to form a homology arm for homologous recombination to delete the transcription regulation site and insert the second target gene to the site of the transcription regulation site. When the second target gene, which was 4HBd, and the transcription regulation site were inserted, the first target gene, which was 025B; the second target gene, which was 4HBd; and the tolC gene were arranged in a 3′ direction from 5′-end.

Next, as a DNA fragment for positive selection, a DNA fragment including, in a 3′ direction from 5′-end, a fifth flanking sequence (SEQ ID NO: 8), a polynucleotide that was the same as the cat2 gene which was a third target gene (SEQ ID NO: 9), a transcription regulation site (SEQ ID NO: 6), and a sixth flanking sequence (SEQ ID NO: 10) was prepared. The fifth flanking sequence (SEQ ID NO: 8) was a polynucleotide that was the same as 50 nucleotides located in a 5′ direction from 3′-end of the 4HBd gene, which was the second target gene. The sixth flanking sequence (SEQ ID NO: 10) was a polynucleotide that was the same as 50 nucleotides located in a 3′ direction from 5′-end of the tolC gene. The fifth flanking sequence and the sixth flanking sequence were designed to form a homology arm for homologous recombination to insert the third target gene, which was cat2, and the transcription regulation site between the 4HBd gene, which was the second target gene, and the tolC gene. When the third target gene, which was cat2, and the transcription regulation site were inserted, the first target gene, which was 025B; the second target gene, which was 4HBd; the third target gene, which was cat2; the transcription regulation site; and the tolC gene were arranged in a 3′ direction from 5′-end.

### 4.2 Successive Insertion of DNA Fragment Including First Target Gene, DNA Fragment Including Second Target Gene, and DNA Fragment Including Third Target Gene to E. coli, and Negative or Positive Selection to Verify Insertion

The DNA fragment including the first target gene, the DNA fragment including the second target gene, and the DNA fragment including the third target gene, which were prepared in Example 4.1, were successively inserted to E. coli by the methods described in Example 1.3 to Example 1.5.

Then, according to the methods described in Example 2.3 to Example 2.5, E. coli, into which the DNA fragment including the first target gene was inserted, was selected by positive selection, E. coli, into which the DNA fragment including the second target gene was inserted, was selected by negative selection, and E. coli, into which the DNA fragment including the third target gene was inserted, was selected by positive selection.

### 4.3 Verification Target Gene Inserted to E. coli Genome

The E. coli, to which a first target gene, a second target gene, and a third target gene were inserted in Example 4.2, was obtained through positive selection or negative selection. To verify the insertion of the first target gene, the second target gene, and the third target gene to the E. coli genome, a PCR was performed by the method described in Example 1.6.

The product obtained by performing a PCR under the conditions shown above underwent electrophoresis in an agarose gel to verify the size of the DNA fragment inserted to the E. coli genome. The result verified that the first target gene, the second target gene, and the third target gene were inserted to the E. coli genome. In addition, the sequence of a DNA fragment was analyzed with respect to E. coli showing a predicted band size to finally verify the insertion of the DNA fragment. The result showed that all the target gene sequences were inserted to the E. coli selected by positive selection and negative selection.

### EXAMPLE 5

### Target Gene Insertion Efficiency According to Orders of Inserting Target Genes

### 5.1. Verification of Target Gene Insertion Efficiency According to Orders of Inserting Target Genes

In the cases where target genes were successively inserted by using an intracellular gene (TolC) as a marker, the target gene insertion efficiency according to the orders of inserting target genes was verified.

Specifically, according to the method described in Example 2, a DNA fragment including the first target gene (025B), a DNA fragment including the second target gene (4HBd), and a DNA fragment including the third target gene (cat2) were successively inserted to E. coli, and the E. coli was selected by a negative selection at an odd-numbered insertion or by a positive selection at an even-numbered insertion.

Additionally, to introduce sucD gene encoding a CoA-dependent succinate-semialdehyde dehydrogenase (SSADH) as a fourth target gene to E. coli genome, a DNA fragment including the sucD gene was prepared, wherein the DNA fragment includes in a 3′ direction from 5′-end a fifteenth flanking sequence (SEQ ID NO: 35), a polynucleotide identical to the sucD gene, which was a fourth target gene (SEQ ID NO: 36), a transcription regulatory region (SEQ ID NO: 6), and a sixteenth flanking sequence (SEQ ID NO: 37). The fifteenth flanking sequence (SEQ ID NO: 35) was a polynucleotide that was the same as 300 nucleotides located in a 5′ direction from the 3′-end of the inserted cat2 gene, which was the third target gene. The sixteenth flanking sequence (SEQ ID NO: 37) was a polynucleotide that was the same as 300 nucleotides located in a 3′ direction from the 5′-end of the tolC gene. When the fourth target gene, which was sucD, was inserted, the first target gene, which was 025B; the second target gene, which was 4HBd; the third target gene, which was cat2; the fourth target gene, which was sucD; a transcription regulatory region; and tolC gene were arranged in a 3′ direction from 5′-end.

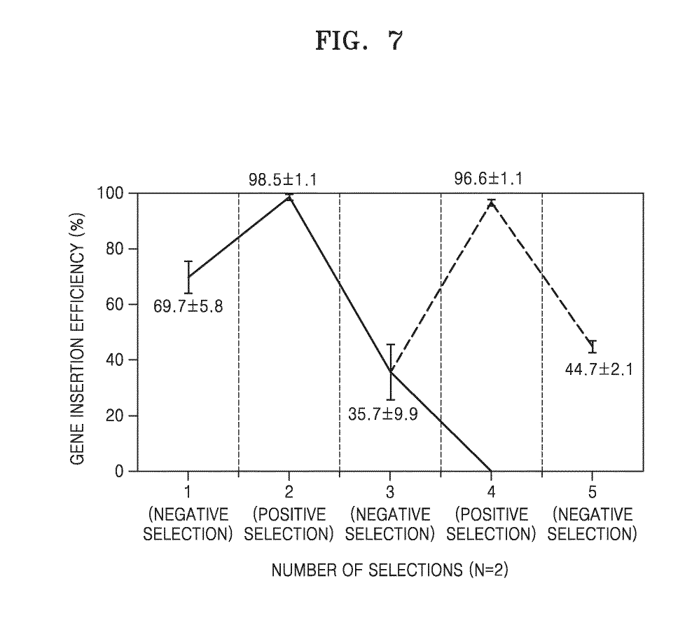

According to the method described in Example 2.3, the genes were inserted to E. coli, and the E. coli was selected by a positive selection. The target gene insertion efficiency was calculated as the efficiency of selecting the target genes, and the result was shown as a solid line in FIG. 7.

As shown in FIG. 7, the efficiency of inserting the first target gene and the second target gene was about 69.7% and about 98.5%, respectively. However, the efficiency of inserting the third target gene and the fourth target gene was significantly decreased to about 35.7% and about 0%, respectively. Therefore, it was verified that the efficiency of inserting a target gene was decreased as the number of times of inserting a target gene was increased.

### 5.2. Verification of Target Gene Insertion Efficiency According to Orders of Inserting Target Genes

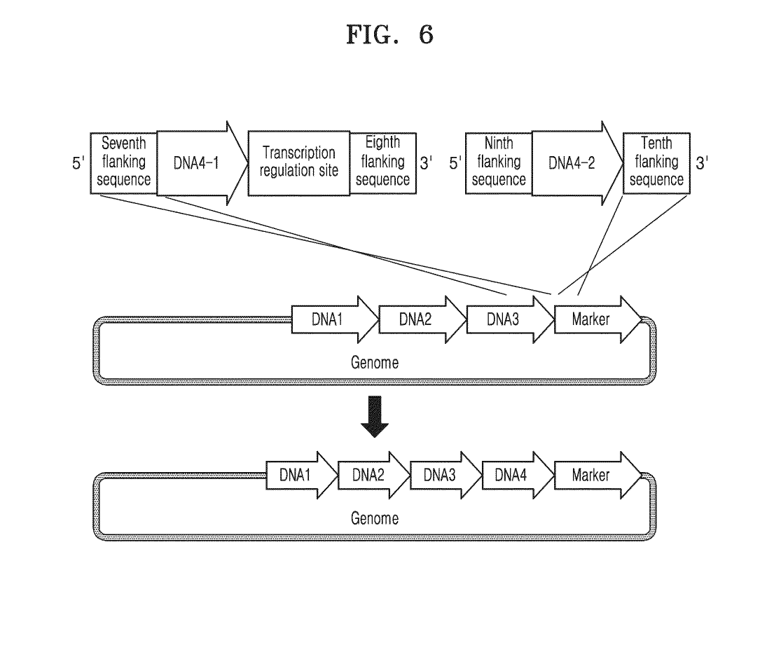

To solve the problem that the efficiency of inserting a target gene was decreased as the number of times of inserting a target gene was increased in successive insertion of target genes, sucD was divided into fragments, as shown in the schematic diagram of FIG. 6, and then inserted to calculate the efficiency of gene insertion.

Specifically, a 5′-end region (sucD-1) of sucD was chosen as a fourth target sequence, and a 3′-end region (sucD-2) of sucD was chosen as a fifth target sequence. As a DNA fragment for inserting the fourth target sequence, a DNA fragment including in a 3′ direction from 5′-end a seventeenth flanking sequence (SEQ ID NO: 38); a fourth target gene, which was sucD-1 (SEQ ID NO: 39); a transcription regulatory region (SEQ ID NO: 6); and an eighteenth flanking sequence (SEQ ID NO: 40) was prepared. As a DNA fragment for inserting the fifth target sequence, a DNA fragment including in a 3′ direction from 5′-end a nineteenth flanking sequence (SEQ ID NO: 41); a fifth target gene, which was sucD-2 (SEQ ID NO: 42); a transcription regulatory region (SEQ ID NO: 6); and an twentieth flanking sequence (SEQ ID NO: 43) was prepared. The sucD-2 (SEQ ID NO: 42) was a region of sucD gene excluding sucD-1 (SEQ ID NO: 39).

The seventeenth flanking sequence (SEQ ID NO: 38) was a polynucleotide that was the same as 300 nucleotides located in a 5′ direction from the 3′-end of the 3′-terminal region of the inserted cat2 gene, which was the third target gene. The eighteenth flanking sequence (SEQ ID NO: 40) was a polynucleotide that was the same as 300 nucleotides located in a 3′ direction from the 5′-end of the tolC gene.

The nineteenth flanking sequence (SEQ ID NO: 41) was a polynucleotide that was the same as 300 nucleotides located in a 5′ direction from the 3′-end of the inserted sucD-1 gene, which was the fourth target gene. The twentieth flanking sequence (SEQ ID NO: 43) was a polynucleotide that was the same as 300 nucleotides located in a 3′ direction from the 5′-end of tolC gene.

When the fourth target gene and the fifth target gene were inserted, the first target gene, which was 025B; the second target gene, which was 4HBd; the third target gene, which was cat2; and the fourth and the fifth target genes, which were sucD and tolC gene were arranged in a 3′ direction from 5′-end.

According to the method described in Example 2.3, the genes were inserted to E. coli, and the E. coli was selected by a positive selection at the insertion of the fourth target gene or by a negative selection at the insertion of the fifth target gene. The target gene insertion efficiency was calculated as the efficiency of selecting the target genes, and the result was shown as a dotted line in FIG. 7.

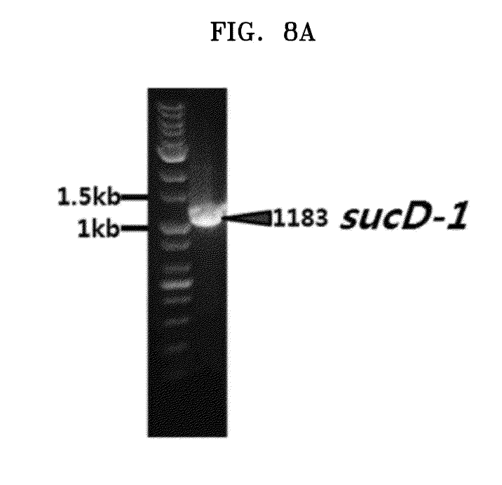

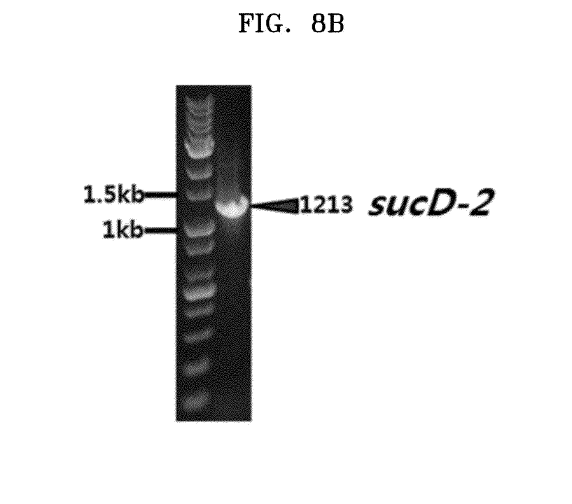

The PCR results of verifying successive insertion of sucD-1 and sucD-2 to a genome were shown in FIG. 8A and 8B, respectively.

As shown in FIG. 7, when the sucD gene was divided into a fourth target gene and a fifth target gene, which were then successively inserted to a genome, the efficiency of inserting the fourth target gene and the fifth target gene was significantly increased to about 96.6% and about 44.7%, respectively. Therefore, it was verified that the efficiency of inserting a target gene may be increased by decreasing the length of an inserted polynucleotide.

As described above, according to the method of inserting a target sequence into a genome of a cell according to an example of the present disclosure, whether a gene is inserted to a microorganism may be rapidly determined by performing positive selection and negative selection based on whether a marker is expressed. In addition, since one marker may be used to successively verify insertion of various genes, more genes may be stably inserted to a genome of a microorganism. In this way, when selecting a microorganism to which a gene is inserted, the efficiency of inserting a DNA fragment may be maximized. Since the method enables to not only effectively detect insertion of a target gene to a genome but also reuse a marker to reduce the size of an inserted gene so that more genes may be inserted to a genome, the method according to one example of the present disclosure has a high industrial applicability.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of inserting a target sequence into a genome of a cell, the method comprising introducing to the cell a first polynucleotide comprising, in a direction from 5′ to 3′, a first flanking sequence, a first region including a first target sequence, and a second flanking sequence,
wherein the genome of the cell comprises, in a direction from 5′ to 3′, a second region comprising a transcription regulation site of a marker encoding region, and a third region comprising a marker encoding region,
wherein the first flanking sequence is homologous with at least two consecutive nucleotides in the second region of the genome of the cell, at least two consecutive nucleotides in a 5′ direction from a 5′-end of the second region, or a combination thereof, and
wherein the second flanking sequence is homologous with at least two consecutive nucleotides in the second region of the genome of the cell, at least two consecutive nucleotides in a 3′ direction from a 5′-end of the third region, or a combination thereof,
whereby the first region of the first polynucleotide is introduced into the genome to provide a first region of the genome.

2. The method of claim 1, wherein the method further comprises, after introducing a first polynucleotide into the cell, introducing a second polynucleotide into the cell, wherein the second polynucleotide comprises, in a direction from 5′ to 3′, a third flanking sequence, a fourth region comprising a second target sequence; a fifth region including a transcription regulation site; and a fourth flanking sequence,
wherein the third flanking sequence is homologous with at least two consecutive nucleotides in a 5′ direction from a 3′ end of the first region of the genome of the cell; and
wherein the fourth flanking sequence is homologous with at least two consecutive nucleotides in a 3′ direction from a 5′-end of the third region of the genome of the cell,
whereby the fourth and fifth regions of the second polynucleotide are introduced into the genome of the cell to provide fourth and fifth regions of the genome.

3. The method of claim 2, wherein the method further comprises, after introducing a second polynucleotide into the cell, introducing a third polynucleotide into the cell, wherein the third polynucleotide comprises, in a direction from 5′ to 3′, a fifth flanking sequence, a sixth region comprising a third target sequence, and a sixth flanking sequence,
wherein the fifth flanking sequence is homologous with at least two consecutive nucleotides in a 5′ direction from 3′-end of the fourth region of the genome of the cell, at least two consecutive nucleotides in the fifth region of the genome, or a combination thereof; and
wherein the sixth flanking sequence is homologous with at least two consecutive nucleotides in a 3′ direction from 5′-end of the third region of the genome of the cell, at least two consecutive nucleotides in the fifth region of the genome, or a combination thereof,
whereby the sixth region of the third polynucleotide is inserted into the genome of the cell to provide a sixth region of the genome.

4. The method of claim 3, wherein the method further comprises, after introducing a third polynucleotide into the cell, introducing a fourth polynucleotide into the cell, wherein the fourth polynucleotide comprises, in a direction from 5′ to 3′, a seventh flanking sequence, a seventh region comprising a region of 5′ direction from 3′-end of a fourth target sequence, and an eighth region comprising a transcription regulatory region, and an eighth flanking sequence,
wherein the seventh flanking sequence is homologous with at least two consecutive nucleotides in a 5′ direction from 3′-end of the six region of the genome of the cell; and
wherein the eighth flanking sequence is homologous with at least two consecutive nucleotides in a 3′ direction from 5′-end of the third region of the genome of the cell,
whereby the seventh and eighth regions of the fourth polynucleotide is introduced into the genome to provide seventh and eighth regions of the genome.

5. The method of claim 4, wherein the method further comprises, after introducing a fourth polynucleotide into the cell, introducing a fifth polynucleotide into the cell, wherein the fifth polynucleotide comprises, in a direction from 5′ to 3′, a ninth flanking sequence, a ninth region comprising a region of 3′ direction from 5′-end of a fourth target sequence, and a tenth flanking sequence,
wherein the fourth target sequence comprises a 3′-end region of the fourth target sequence and a 5′-end region of the fourth target sequence,
wherein the ninth flanking sequence is homologous with at least two consecutive nucleotides in a 5′ direction from 3′-end of the seventh region of the genome of the cell, at least two consecutive nucleotides in the eighth region, or a combination thereof; and
wherein the tenth flanking sequence is homologous with at least two consecutive polynucleotides in a 3′ direction from 5′-end of the third region of the genome of the cell, at least two consecutive nucleotides in the eighth region, or a combination thereof
whereby the ninth region of the fourth polynucleotide is introduced into the genome to provide a ninth region of the genome.

6. The method of claim 1, wherein the transcription regulation site is a promoter, an enhancer, an insulator, a silencer, a polynucleotide encoding a transcription factor, or a combination thereof.

7. The method of claim 1, wherein the marker is a membrane protein, a glycolysis metabolism-related protein, a DNA biosynthesis-related protein, or a combination thereof.

8. The method of claim 1, wherein the method further comprises selecting a cell in which a marker encoded by the third region of the genome is not expressed to perform a negative selection of a cell in which a first target sequence is inserted to a genome of a cell.

9. The method of claim 2, wherein the method further comprises selecting a cell in which a marker encoded by the third region of the genome is expressed to perform a positive selection of a cell in which a second target sequence is inserted to a genome of a cell.

10. The method of claim 3, wherein the method further comprises selecting a cell in which a marker encoded by the third region of the genome is not expressed to perform a negative selection of a cell in which a third target sequence is inserted to a genome of a cell.

11. A method of inserting a target sequence into a genome of a cell, the method comprising introducing a first polynucleotide comprising, in a direction from 5′ to 3′, a first flanking sequence, a first region comprising a first target sequence, a second region comprising a transcription regulation site, a third region comprising a marker encoding region, and a second flanking sequence,
wherein the first flanking sequence and the second flanking sequence are homologous with two or more consecutive nucleotides in the direction of a 5′-end of an insertion site of the genome of the cell, or two or more consecutive nucleotides in the direction of a 3′-end of an insertion site of the genome of the cell
whereby the first, second, and third regions of the first polynucleotide are inserted into the genome of the cell to provide first, second, and third regions of the genome.

12. The method of claim 11, wherein the method further comprises, after introducing a first polynucleotide to a cell, introducing a second polynucleotide into the cell, wherein the second polynucleotide comprises, in a direction from 5′ to 3′, a third flanking sequence, a fourth region comprising a second target sequence, and a fourth flanking sequence,
wherein the third flanking sequence is homologous with at least two consecutive nucleotides in a 5′ direction from 3′-end of the first region of the genome of the cell, at least two consecutive nucleotides in the second region, or a combination thereof, and
wherein the fourth flanking sequence is homologous with at least two consecutive nucleotides in the second region of the genome of the cell, at least two consecutive nucleotides in a 3′ direction from 5′-end of the third region, or a combination thereof,
whereby the fourth region of the second polynucleotide is inserted into the genome of the cell to provide a fourth region of the genome.

13. The method of claim 12, wherein the method further comprises, after introducing a second polynucleotide to a cell, introducing a third polynucleotide into the cell, wherein the third polynucleotide comprises, in a direction from 5′ to 3′, a fifth flanking sequence, a sixth region comprising a third target sequence; a fifth region comprising a transcription regulation site; and a sixth flanking sequence,
wherein the fifth flanking sequence is homologous with at least two consecutive nucleotides in a 5′ direction from a 3′-end of the fourth region of the genome of the cell; and
wherein the sixth flanking sequence is homologous with at least two consecutive nucleotides in a 3′ direction from a 5′-end of the third region of the genome of the cell,
whereby the fifth and sixth regions of the third polynucleotide are inserted into the genome of the cell to provide fifth and sixth regions of the genome.

14. The method of claim 13, wherein the method further comprises, after introducing a third polynucleotide into the cell, introducing a fourth polynucleotide into the cell, wherein the fourth polynucleotide comprises, in a direction from 5′ to 3′, a seventh flanking sequence, a seventh region comprising a region of 5′ direction from 3′-end of a fourth target sequence, and an eighth flanking sequence,
wherein the seventh flanking sequence is homologous with at least two consecutive nucleotides in a 5′ direction from 3′-end of the sixth region of the genome of the cell, at least two consecutive nucleotides in the fifth region, or a combination thereof; and
wherein the eighth flanking sequence is homologous with at least two consecutive nucleotides in a 3′ direction from 5′-end of the third region of the genome of the cell, at least two consecutive nucleotides in the fifth region, or a combination thereof,
whereby the seventh region of the fourth polynucleotide is inserted into the genome of the cell to provide a seventh region of the genome.

15. The method of claim 17, wherein the method further comprises, after introducing a fourth polynucleotide into the cell, introducing a fifth polynucleotide into the cell, wherein the fifth polynucleotide comprises, in a direction from 5′ to 3′, a ninth flanking sequence, an eighth region comprising a region of 3′ direction from 5′-end of a fourth target sequence, a ninth region comprising a transcription regulatory region, and a tenth flanking sequence,
wherein the fourth target sequence comprises a 3′-end region of the fourth target sequence and a 5′-end region of the fourth target sequence,
wherein the ninth flanking sequence is homologous with at least two consecutive nucleotides in a 5′ direction from 3′-end of the seventh region of the genome of the cell; and
wherein the tenth flanking sequence is homologous with at least two consecutive nucleotides in a 3′ direction from 5′-end of the third region of the genome of the cell,
whereby the eighth and ninth regions of the fourth polynucleotide is introduced into the genome to provide eighth and ninth regions of the genome.

16. The method of claim 11, wherein the transcription regulation site is a promoter, an enhancer, an insulator, a silencer, a polynucleotide encoding a transcription factor, or a combination thereof.

17. The method of claim 11, wherein the marker is a membrane protein, a glycolysis metabolism-related protein, a DNA biosynthesis-related protein, or a combination thereof.

18. The method of claim 11, wherein the method further comprises selecting a cell in which the marker is expressed to perform a positive selection of a cell in which a first target sequence is inserted to a genome of a cell.

19. The method of claim 12, wherein the method further comprises selecting a cell in which the marker is not expressed to perform a negative selection of a cell in which a second target sequence is inserted to a genome of a cell.

20. The method of claim 13, wherein the method further comprises selecting a cell in which the marker is expressed to perform a positive selection of a cell in which a third target sequence is inserted to a genome of a cell.


SEQUENCE LISTING (43 sequences)

SEQ ID NO: 1 - DNA, 50 bp, Artificial Sequence
  Synthetic (flanking sequence of pylT,S)
catcgtttttgccaaatgtaacgggcaggttgtctggcttaagcattgtt

SEQ ID NO: 2 - DNA, 1638 bp, Artificial Sequence
  Synthetic (pylT,S)
aaaaaaatattctcaacataaaaaactttgtgtaatacttgtaacgctgaattcggaaacctgatcatgtagatcgaatggactctaaatccgttcagccgggttagattcccggggtttccgccaatgcatatccttaatccttagcgaaagctaaggattttttttggatcggaattaattcccggtttaaaccggggatctcgatcccgcgaaattaatacgactcactataggggaattgtgagcggataacaattcccctctagaaataattttgtttaactttaagaaggagagatctatatggataaaaagccgctcgacacgcttatttcggcgacgggcctgtggatgtcccgcacgggtatgattcacaaaattaaacaccacgaagtgtcgcgttcaaaaatctatatcgaaatggcatgcggtgagcgtctggttgtgaacaacagccgtagcagccgtactgcacgggcactccgtcatcacaagtatcgaaagacgtgccggcattgccgagtatctgatgaagacattaacaactttcttactaaaacctccgaagaaaaaacaacagtcaaagttaaggtagtctccgctccccgagtacgcaaagcgatgccaaaatccgttgcacgtgccccgaaaccgctagaggctactgctcaagttccgctttcggggtcaaagccggcgccggccactccggtttcggcacccgcacaggcgcctgctccgtccactgggtccgcgtcagctacttctgcatcagcacagcggatggcgaattcggccgcagctcctgcggcaccggtgcccacatccgcaccagcactgactaagggccaactagaccgcctcgaaggtctcctctcaccaaaggatgaaatttctctcgattccgagaaaccattccgagaactcgagtccgaactgctctcccgccggaaaaaagacctgaagcgtatttatgcggaagaacgcgaaaactatctcggtaagcttgagcgcgaaatcacgaagttcttcgttgaccgtgggtttctcgaaatcaaatctccgattcttatccctgctgaatatgtggaacgtatgggcatcaactcggacacagagctgtcgaagcaagtattccgtatcgacaaaaacttctgccttcgacccatgctggcaccaaacctatacaactacctacggaaactagaccgtgctctacccgatccgattaaaattttcgagattggaccatgctatcgaaaagaatctgatggaaaagaacacctagaagagtttacgatgctcaacttctgccagatgggttccggctgcacgcgagagaatctcgaggcgatcatcacagaattcctgaatcatctaggcattgattttgaaattattggtgattcttgcatggtgtatggaaatacgctagatgtcatgcacgatgacctagagctttcttcagctgtagttgggccggttccgttggaccgtgagtggggaatcgataaaccgtggatcggggcaggctttggactagagcgtcttctgaaggttatgcatggattcaaaaatattaagcgagccgcccgctctgaatcatattataatggaatttcgaccaacctttaa

SEQ ID NO: 3 - DNA, 50 bp, Artificial Sequence
  Synthetic (flanking sequence of pylT,S)
ctgaacccagaaaggctcaggccgataagaatggggagcaatttcttcat

SEQ ID NO: 4 - DNA, 50 bp, Artificial Sequence
  Synthetic (flanking sequence of pylB)
gagccgcccgctctgaatcatattataatggaatttcgaccaacctttaa

SEQ ID NO: 5 - DNA, 1053 bp, Artificial Sequence
  Synthetic (pylB)
atgattaaaaagatggcgactgaagacctggaccgtttcggggaaaaaattattgaaggtttccaactttctgacgatgacctacgtgctcttctgtctctggaatctgaggaggagctcgagaaactttactatgtggcacgtaaagtgcgtaaccactacttcggcaaccgtgtctttcttaattgcttcatctacttttctacatattgtaaaaaccagtgcgccttctgttattataattgtaagaacgaaatcaaccgatatcgtctttcacgtgaagagattaaagaaacctgtaaagcgctgaagggcgccggtttccatatgatcgacctaacgatgggcgaggacccgtactattatgaggagccggatcgtttcgttgagcttgtacaaatggtcaagaatgagctaggacttcccattatgatctccccgggcgtaatggataatgcgaccctgctgaaagcccgtgaaaagggagctaacttctttgccctataccaggaaacatatgatcaagagctctatgagaaactgcgcgttggccaatcgtttaaaggtcggtacaacactcgacgttttgctaaggaacaaggatactgcatcgaagatggcatcctgacgggagttgggaacgatattgaatcgacgatcaagtcgcttcgtggaatgaagtctaacgacccagacatggtccgagtaatgactttccttccacaggagggaacaccgcttgagcgttttaaggagaactcgaacctgtcggaactgaaaatcattgcgattctacggctaatctttcctaagtgcctgatccccgcctctcttgaccttgagggcattgatggaatggtgcaccgtctcaatgcaggcgcaaatattgttacttcaatcctcccttcagattctaagctggaaggtgttgcgaactatgaccgtgatcttgaagagcgagatcgcgatattaagtcagtcattaagcgacttgaaggtatggggatggaacctgcgccacaagctgagttcgaaacagttctccgatgttaa

SEQ ID NO: 6 - DNA, 58 bp, Artificial Sequence
  Synthetic (transcription regulation site)
taatacgactcactataggggaattgtgagcgtttaactttaagaaggagatatacat

SEQ ID NO: 7 - DNA, 50 bp, Artificial Sequence; identical to SEQ ID NO: 3
  Synthetic (flanking sequence of pylB)

SEQ ID NO: 8 - DNA, 50 bp, Artificial Sequence
  Synthetic (flanking sequence of pylC,D)
ttaacatcggagaactgtttcgaactcagcttgtggcgcaggttccatcc

SEQ ID NO: 9 - DNA, 2202 bp, Artificial Sequence
  Synthetic (pylC,D)
atgaaaactatttgtctaattggtggaaaactacagggttttgaggctgcgtatctgtcgaaaaaagcggatatgaaagttgttgtcattgacaagaacccccaggccctgattcgtaattatgcggatgaatttcactgtttcgatgttatcaaggagccggagaagcttctagaaatctcaaaaaacgtagatgccatcctacctgtcaacgagaacctggaatgtatcaagtttctatcgtctgtcaaggaggaattctcatgcccggttctcttcgactttgaagcttatcgtatctctcgtgacaaaaagaagtctaaggaatattttcgatcaattggtatcccgacacctcaggataaacccggacgcccgccttatttcgttaagcctccgtgtgaatcgtcatcggtcggtgcccgtatcatctatgatgaagaggaactaggagaactggaaccgggcatgctggtcgaggaatacgttgaaggcgaggtcatttcgctcgaggtaatcggggacgggactcatttcgcagtcgtcaaggagacactaatccatatcgatcgtacatatgattgtcacatggtaacaccactgccttcagacctatctttccgtgaaatcagctactctcttgcggcaaatcttcctctgaaaggtattatggatgtggaagcgatttctggaccccagggacttaaggttatcgaaattgattcacgatttccttcacaaacccctaccgctgtgtattactcatctggcgttaatttaatcgaactgctgttccgtgcttttggtgagggagtcgaggaggttaagacacttccagaagatcggtactgtatctacgaacatctaatgcttgccgaaaatggagctctcattcccgtcggcgagcaagtactctcgatgggaaacgattacggcaaatactatgaagagccgggaattgaaatttttctttgtcggggagaggaccctgtatttacgctcgttttttggggtaaggaccgggaggaggctgaaaacaaaaaacgcaccggcctgctgattctcaaagaccgattcggtgctgctgtttaactgcaggaaggggatccggctgctaacaaagcccgaaaggaagctgagttggctgctgccaccgctgagcaataactagcataaccccttggggcctctaaacgggtcttgaggggttttttgctgaaaggaggaactatatccggataactacgtcaggtggcacttttcggggaaatgtgcgcggaacccggatcggaattaattcccggtttaaaccggggatctcgatcccgcgaaattaatacgactcactataggggaattgtgagcggataacaattcccctctagaaataattttgtttaactttaagaaggagaccggtatatggctctccttacaccggaggacctagaaaatattaaccggcaactacacgaagcggattcgacggtgcgccgtgtcactggacttgacatcaagggagtgtgcaaagatttttacggtaccacgccatgctgcgaaaaggttggcatcgttccggtgacgtccgggaacgggattatcggtaacttctcggaatcactccgagcaattactgaatacttcggtttcgattcattcgtaacaacgatgcctgacgtatctggatactacgaagcagtacggaacggcgcacaaatcattcttatggcggatgacaacacctttctggcgcacaacctcaagaacggaaaaattgcgaataatcagccttgcaccgggatcatttatgcagagatcgcttcgcgatatatgaaagccgactcgaaagacgtactggctgtaggactaggtaaggtagggtttccaggcgcagcgcatttggttaacaaggggtttcgcgtgtacgggtacgatgccgacaaggctctcctcgaaaaaaccgtttcagatctcggtatcacaccatttgagccagagaaccctcgccgtttttccattatttttgaggctaccccctgtgcggacactgtcccggaaagcgtgatctccgacaattgtgtcatctcaactccaggaattccctgcgcaatttcgcgtgaactccaggagaagtatggggtagaactaatcatggagccccttggtatcggcactgcctctatgctgtattcgatcctgtaa

SEQ ID NO: 10 - DNA, 50 bp, Artificial Sequence; identical to SEQ ID NO: 3
  Synthetic (flanking sequence of pylC,D)

SEQ ID NO: 11 - DNA, 20 bp, Artificial Sequence
  Synthetic (pylT,S  Fwd primer)
aagccgcgataaagtgtttc

SEQ ID NO: 12 - DNA, 16 bp, Artificial Sequence
  Synthetic (pylT,S  Rev primer)
gttctcggcctggctc

SEQ ID NO: 13 - DNA, 23 bp, Artificial Sequence
  Synthetic (pylB  Fwd primer)
catggtgtatggaaatacgctag

SEQ ID NO: 14 - DNA, 16 bp, Artificial Sequence
  Synthetic (pylB  Rev primer)
gttctcggcctggctc

SEQ ID NO: 15 - DNA, 23 bp, Artificial Sequence
  Synthetic (pylC,D  Fwd primer)
cttcagattctaagctggaaggt

SEQ ID NO: 16 - DNA, 16 bp, Artificial Sequence
  Synthetic (pylC,D  Rev primer)
gttctcggcctggctc

SEQ ID NO: 17 - DNA, 300 bp, Artificial Sequence
  Synthetic (flanking sequence of 025B)
atcatcccggcaaccatctccagtagccaaggggtttcgctggtgtcgtacgcggcaatccgaatctgctcaatcagcacaacttcatcacgcactgggtcaaagggtagcaagactgcggcgtgaccgcgctcaaaaatttcccgccgcacctcatgactcatttgcccgttgaatagacgatgacgaaatctataaagatctaatgaaaaaaagccgcgataaagtgtttctcgtgcaataatttctacatcgtttttgccaaatgtaacgggcaggttgtctggcttaagcattgtt

SEQ ID NO: 18 - DNA, 1407 bp, Artificial Sequence
  Synthetic (025B)
atgaataaagacacactgatccctacaactaaagatttaaaagtaaaaacaaatggtgaaaacattaatttaaagaactacaaagataatagcagttgtttcggcgtattcgaaaatgttgaaaatgctatcagcagcgctgtacacgcacaaaagatattatcgctgcattatacaaaagagcaacgtgaaaaaatcatcactgagatacgtaaggccgcattacaaaataaagaggtgctggctacaatgattctggaagaaacacatatgggacgttatgaggataaaatattaaaacatgaactggtagctaaatatactcctggtacagaagatttaactactactgcctggagcggtgataatggtctgacagttgtagaaatgtctccatatggtgttattggtgcaataactccttctaccaatccaactgaaactgtaatttgtaatagcattggcatgattgctgctggaaatgctgtagtatttaacggacacccatgcgctaaaaaatgtgttgcctttgctgttgaaatgatcaataaggcaattattagctgtggcggtccggaaaatctggtaacaactataaaaaatccaaccatggagtctctggatgccattattaagcatccttcaataaaactgctttgcggaactggcggtccaggaatggtaaaaaccctgttaaattctggtaagaaagctattggtgctggtgctggaaatccaccagttattgtcgatgatactgctgatattgaaaaggctggtcgtagcatcattgaaggctgttcttttgataataatttaccttgtattgcagaaaaagaagtatttgtttttgagaatgttgcagatgatttaatatctaacatgctgaaaaataatgctgtaattatcaatgaagatcaggtatcaaaattaatcgatttagtattacaaaaaaataatgaaactcaagaatactttatcaacaaaaaatgggtaggtaaagatgcaaaattattcctcgatgaaatcgatgttgagtctccttcaaatgttaaatgcattatctgcgaagtgaatgccaatcatccatttgttatgacagaactgatgatgccaatattgccaattgtgcgcgttaaagatatcgatgaagctattaaatatgcaaagattgcagaacaaaatagaaaacatagtgcctatatttatagcaaaaatatcgacaacctgaatcgctttgaacgtgaaatcgatactactatttttgtaaagaatgctaaatcttttgctggtgttggttatgaagcagaaggatttaccactttcactattgctggatctactggtgagggcataacctctgcacgtaattttacccgccaacgtcgctgtgtactggccggctaa

SEQ ID NO: 19 - DNA, 300 bp, Artificial Sequence
  Synthetic (flanking sequence of 025B)
catatcaaaaatggattgagttaactgcaaggacgcactggtcgcgttagagttgatgccgttcgcgtcgcggtagccgttgctataggtgtaatctgcacctaaacctagctgtggcagtaatggactgcgcgcttcattaattttttcaaaggcagcatcacgatcggcggcagacttacgcaattccgggttactaaggcgtgcttgctgataaacttgcatcaggttctcggcctggctcaacgaactgaacccagaaaggctcaggccgataagaatggggagcaatttcttcat

SEQ ID NO: 20 - DNA, 300 bp, Artificial Sequence
  Synthetic (flanking sequence of 4HBd)
atgatgccaatattgccaattgtgcgcgttaaagatatcgatgaagctattaaatatgcaaagattgcagaacaaaatagaaaacatagtgcctatatttatagcaaaaatatcgacaacctgaatcgctttgaacgtgaaatcgatactactatttttgtaaagaatgctaaatcttttgctggtgttggttatgaagcagaaggatttaccactttcactattgctggatctactggtgagggcataacctctgcacgtaattttacccgccaacgtcgctgtgtactggccggctaa

SEQ ID NO: 21 - DNA, 1116 bp, Artificial Sequence
  Synthetic (4HBd)
atgcaacttttcaaactcaagagtgtaacacatcactttgacacttttgcagaatttgccaaggaattctgtcttggagaacgcgacttggtaattaccaacgagttcatctatgaaccgtatatgaaggcatgccagctcccctgccattttgttatgcaggagaaatatgggcaaggcgagccttctgacgaaatgatgaataacatcttggcagacatccgtaatatccagttcgaccgcgtaatcggtatcggaggaggtacggttattgacatctctaaacttttcgttctgaaaggattaaatgatgtactcgatgcattcgaccgcaaaatacctcttatcaaagagaaagaactgatcattgtgcccacaacatgcggaacgggtagcgaggtgacgaacatttctatcgcagaaatcaaaagccgtcacaccaaaatgggattggctgacgatgccattgttgcagaccatgccatcatcatacctgaacttctgaagagcttgcctttccacttctacgcatgcagtgcaatcgatgctcttatccatgccatcgagtcatacgtatctcctaaagccagtccatattctcgtctgttcagtgaggcggcttgggacattatcctggaagtattcaagaaaatcgccgaacacggccctgaataccgcttcgaaaagctgggagaaatgatcatggccagcaactatgccggtatagccttcggaaatgcaggagtaggagccgtccacgcactatcctacccgttgggaggcaactatcacgtgccgcatggagaagcaaactatcagttcttcacagaggtattcaaagtataccaaaagaagaatcctttcggctatatagtcgaactcaactggaagctctccaagatactgaactgccagcccgaatacgtatatccgaagctggatgaacttctcggatgccttcttaccaagaaacctttgcacgaatacggcatgaaggacgaagaggtaagaggctttgcggaatcagtgcttaagacacagcaaagattgctcgccaacaactacgtagagcttactgtagatgagatcgaaggtatctacagaagactctactaa

SEQ ID NO: 22 - DNA, 300 bp, Artificial Sequence; identical to SEQ ID NO: 19
  Synthetic (flanking sequence of 4HBd)

SEQ ID NO: 23 - DNA, 300 bp, Artificial Sequence
  Synthetic (flanking sequence of cat2)
cagttcttcacagaggtattcaaagtataccaaaagaagaatcctttcggctatatagtcgaactcaactggaagctctccaagatactgaactgccagcccgaatacgtatatccgaagctggatgaacttctcggatgccttcttaccaagaaacctttgcacgaatacggcatgaaggacgaagaggtaagaggctttgcggaatcagtgcttaagacacagcaaagattgctcgccaacaactacgtagagcttactgtagatgagatcgaaggtatctacagaagactctactaa

SEQ ID NO: 24 - DNA, 1296 bp, Artificial Sequence
  Synthetic (cat2)
atgcaatggcaagaactttaccgtcagcgcgtttgctctgcagacgaagctgtcgtggactctcttaaaccgggaacgaaagttgtattcggtcatgctgctgctgcgcctgtccgtttctctcaggctatgtaccgccagcgtgaaaagttggagaatatcacagttttccacatgttgtatttcggcgacgcgccgcaccttgctcccgaaatgcgttcgcatgtacacccgactctcaacttccttgagggcaactcccgtccggcaagccgtgaccgtcgtgtcgatttcattccctgccacttccacgaggtaccggaactgtttcgtcagggattctttccattggatgtagccgtagtgcaggtatctactcctaacgaagagggttattgctctttcggagtttcctgcgactacacaaaggctgccgccgagtgcgctccggtagtagtagccgaggtgaacaagcaaatgccattcatcggtggtgaaaacctgattcacatctccaaactgacccatatcatcgaagtggacgagccgattgcagaagtattgcctcctgctatcagcgaccttgaactgaggataggtcagaattgtgcctcactgatcaaagacggcgataccctccagttgggtatcggcggtatccccgacgctgtgttgcgtgcattggaagggcataaagatctcggtattcacacggaaatgtttaccgacggggtgatgcgtatgattcgcaaggggattatcaacgggaagaaaaaaacattgcatcccgaaaaagtcgttacctcgctaatcttcggatcgaaagaattgtacgattttgtcaataacaatccggtgatagaatgctatccggtggattatatcaacaaccccgatgttatcggtaagaatgaccgcatggtttctatcaattcctgcttggagatggatctcatggggcaggcagcttctgagtcgatcgggtacgaacagttcagtggatccggaggtcaagtcgatttccttcgtggggccaagcgttccaagggaggaatctccattatggctttccccagtacggccaagaaagggactgagagtcgcatcgttcccattctgaaagagggtgcttgtgtcacgaccggccgtaacgaagtggactatgtggtgacggaatatggcgtagcgcgtctgcgtggcgcaacgcttcgtcagcgtgctgaagccttgactgctatagcacatcccgatttccgaccggcccttgaggaggaaatccgccgacgcttcgaataa

SEQ ID NO: 25 - DNA, 300 bp, Artificial Sequence; identical to SEQ ID NO: 19
  Synthetic (flanking sequence of cat2)

SEQ ID NO: 26 - DNA, 22 bp, Artificial Sequence
  Synthetic (025B  Fwd primer)
gtttggtccgtttgactatcag

SEQ ID NO: 27 - DNA, 19 bp, Artificial Sequence
  Synthetic (025B  Rev primer)
aggtttgctgatcggtctg

SEQ ID NO: 28 - DNA, 26 bp, Artificial Sequence
  Synthetic (4HBd  Fwd primer)
aaatgttaaatgcattatctgcgaag

SEQ ID NO: 29 - DNA, 19 bp, Artificial Sequence
  Synthetic (4HBd  Rev primer)
aggtttgctgatcggtctg

SEQ ID NO: 30 - DNA, 30 bp, Artificial Sequence
  Synthetic (cat2  Fwd primer)
ggagaaatgatcatggccagcaactatgcc

SEQ ID NO: 31 - DNA, 19 bp, Artificial Sequence
  Synthetic (cat2  Rev primer)
aggtttgctgatcggtctg

SEQ ID NO: 32 - DNA, 50 bp, Artificial Sequence; identical to SEQ ID NO: 1
  Synthetic (flanking sequence 13)

SEQ ID NO: 33 - DNA, 1482 bp, Artificial Sequence
  Synthetic (TolC gene)
atgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 34 - DNA, 50 bp, Artificial Sequence
  Synthetic (flanking sequence 14)
atctttacgttgccttacgttcagacggggccgaagccccgtcgtcgtca

SEQ ID NO: 35 - DNA, 300 bp, Artificial Sequence
  Synthetic (flanking sequence of sucD)
agtggcaccgggggtcaagtagattatgtccgcggggcagcttggtctaaaaacggcaaaagcatcatggcaattccctcaacagccaaaaacggtactgcatctcggatagttcctataattgcagagggcgctgctgtaacaaccctccgcaacgaagtcgactacgttgttacggaatatgggatagcacagttaaaaggtaagagtttgcgtcagcgcgcagaagctcttattgcgatagcccacccggactttagagaggaactgacgaagcatctgcgcaaacgttttggttaa

SEQ ID NO: 36 - DNA, 1356 bp, Artificial Sequence
  Synthetic (sucD)
atggaaataaaagagatggtgtcgttggcaaggaaagctcagaaggaatatcaagcgacccataatcaagaagcagttgataacatttgccgagctgcagcaaaagtgatttatgaaaatgcagctatactggctcgcgaagcagtagacgaaaccggcatgggcgtatatgaacataaagtggccaagaatcaggggaaatccaaaggcgtctggtacaatttgcacaataaaaaatcgatcggtatcttaaatatagacgagagaaccgggatgatcgagatagcaaaacctatcggggttgttggagccgtaaccccgacgacaaacccgattgtgactccaatgagcaacatcatttttgcccttaagacatgcaatgccattattatcgccccacatcccagatccaaaaaatgctcagcacatgcagttcgtctgataaaggaagcaatcgctccgtttaatgtcccggagggaatggttcagatcattgaagagcccagcatcgagaaaactcaggaactaatgggcgccgtggatgtggtagttgcgacgggtggtatgggtatggtgaaatctgcatattcttcagggaagccttcttttggtgtaggagccggtaacgttcaagtgatcgtggatagtaatatcgattttgaagctgcggcagaaaaaattatcaccggccgtgctttcgacaatgggatcatctgttcaggcgaacagagtatcatctacaacgaagctgacaaggaagctgtcttcacagccttccgcaaccatggtgcatatttttgtgatgaagcggagggagatcgggcccgtgctgcgatttttgagaatggcgccatcgcgaaagatgtagtcggccagagcgttgcctttatcgcgaagaaagcaaatatcaatataccggagggtacccgtattctggttgttgaagctcgcggcgtcggagcagaggatgtcatatgtaaggaaaaaatgtgtccagttatgtgcgccttaagctacaagcacttcgaggaaggtgtagaaatcgcacgtacgaacttggccaacgaaggtaacggccatacctgtgcgatccattccaacaatcaggcgcatatcatactggcaggttcagaactgacggtttcgcggatcgtggtcaatgcgccgagtgccactacagcaggcggtcacatccaaaatggtctggcagtgacaaatacgctcggatgcgggagttggggtaataactctatctccgagaactttacttataaacacctgttaaacattagccgcatagcgccgcttaattcaagcattcacattcctgatgacaaagagatctgggaactctaa

SEQ ID NO: 37 - DNA, 300 bp, Artificial Sequence; identical to SEQ ID NO: 19
  Synthetic (flanking sequence of sucD)

SEQ ID NO: 38 - DNA, 300 bp, Artificial Sequence; identical to SEQ ID NO: 35
  Synthetic (flanking sequence of sucD-1)

SEQ ID NO: 39 - DNA, 665 bp, Artificial Sequence
  Synthetic (sucD-1)
atggaaataaaagagatggtgtcgttggcaaggaaagctcagaaggaatatcaagcgacccataatcaagaagcagttgataacatttgccgagctgcagcaaaagtgatttatgaaaatgcagctatactggctcgcgaagcagtagacgaaaccggcatgggcgtatatgaacataaagtggccaagaatcaggggaaatccaaaggcgtctggtacaatttgcacaataaaaaatcgatcggtatcttaaatatagacgagagaaccgggatgatcgagatagcaaaacctatcggggttgttggagccgtaaccccgacgacaaacccgattgtgactccaatgagcaacatcatttttgcccttaagacatgcaatgccattattatcgccccacatcccagatccaaaaaatgctcagcacatgcagttcgtctgataaaggaagcaatcgctccgtttaatgtcccggagggaatggttcagatcattgaagagcccagcatcgagaaaactcaggaactaatgggcgccgtggatgtggtagttgcgacgggtggtatgggtatggtgaaatctgcatattcttcagggaagccttcttttggtgtaggagccggtaacgttcaagtgatcgtggatagtaatatcgattttgaagc

SEQ ID NO: 40 - DNA, 300 bp, Artificial Sequence; identical to SEQ ID NO: 19
  Synthetic (flanking sequence of sucD-1)

SEQ ID NO: 41 - DNA, 300 bp, Artificial Sequence
  Synthetic (flanking sequence of sucD-2)
ccttaagacatgcaatgccattattatcgccccacatcccagatccaaaaaatgctcagcacatgcagttcgtctgataaaggaagcaatcgctccgtttaatgtcccggagggaatggttcagatcattgaagagcccagcatcgagaaaactcaggaactaatgggcgccgtggatgtggtagttgcgacgggtggtatgggtatggtgaaatctgcatattcttcagggaagccttcttttggtgtaggagccggtaacgttcaagtgatcgtggatagtaatatcgattttgaagc

SEQ ID NO: 42 - DNA, 691 bp, Artificial Sequence
  Synthetic (sucD-2)
tgcggcagaaaaaattatcaccggccgtgctttcgacaatgggatcatctgttcaggcgaacagagtatcatctacaacgaagctgacaaggaagctgtcttcacagccttccgcaaccatggtgcatatttttgtgatgaagcggagggagatcgggcccgtgctgcgatttttgagaatggcgccatcgcgaaagatgtagtcggccagagcgttgcctttatcgcgaagaaagcaaatatcaatataccggagggtacccgtattctggttgttgaagctcgcggcgtcggagcagaggatgtcatatgtaaggaaaaaatgtgtccagttatgtgcgccttaagctacaagcacttcgaggaaggtgtagaaatcgcacgtacgaacttggccaacgaaggtaacggccatacctgtgcgatccattccaacaatcaggcgcatatcatactggcaggttcagaactgacggtttcgcggatcgtggtcaatgcgccgagtgccactacagcaggcggtcacatccaaaatggtctggcagtgacaaatacgctcggatgcgggagttggggtaataactctatctccgagaactttacttataaacacctgttaaacattagccgcatagcgccgcttaattcaagcattcacattcctgatgacaaagagatctgggaactctaa

SEQ ID NO: 43 - DNA, 300 bp, Artificial Sequence; identical to SEQ ID NO: 19
  Synthetic (flanking sequence of sucD-2)